(19) United States
(12) Patent Application Publication
(10) Pub. No.: US 2005/0031625 A1
(43) Pub. Date: Feb. 10, 2005

(54) Methods and compositions for conversion of antibody activity

(75) Inventors: Nehal Mohamed, Budd Lake, NJ (US); George L. Spitalny, Cheshire, CT (US); Leslie S. Casey, New York, NY (US)

Correspondence Address: LAHIVE & COCKFIELD, LLP., 28 STATE STREET, BOSTON, MA, 02109, US

(73) Assignee: EluSys Therapeutics, Inc., Pine Brook, NJ (US)

(21) Appl. No.: 10/812,636

(22) Filed: Mar. 29, 2004

Related U.S. Application Data
(60) Provisional application No. 60/458,468, filed on Mar. 28, 2003.

(51) Int. Cl. A61K 39/40; C07K 16/46

(52) U.S. Cl. 424/164.1; 424/178.1; 530/388.4

(57) ABSTRACT

The present invention provide a bispecific molecule comprising an antibody that binds a C3b-like receptor linked to one or more non-neutralizing antigen-binding antibodies or fragments thereof. The present invention also provides methods to identify non-neutralizing antibodies, and particularly, to identify enhancing antibodies. Methods of producing such bispecific molecules and their therapeutic and/or prophylactic uses are also provided by the present invention

### RELATED APPLICATIONS

This application claims the benefit of U.S. Provisional Patent Application No. 60/458,468, filed on Mar. 28, 2003, tilted “METHODS AND COMPOSITIONS FOR CONVERSION OF ANTIBODY ACTIVITY.” The entire contents of this application are hereby incorporated herein by reference.

### BACKGROUND OF THE INVENTION

Primate erythrocytes, or red blood cells (RBC's), play an essential role in the clearance of antigens from the circulatory system. The formation of an immune complex in the circulatory system activates the complement factor C3b in primates and leads to the binding of C3b to the immune complex. The C3b/immune complex then binds to the type 1 complement receptor (CR1), a C3b receptor, expressed on the surface of erythrocytes via the C3b molecule attached to the immune complex. The immune complex is then chaperoned by the erythrocyte to the reticuloendothelial system (RES) in the liver and spleen for neutralization. The RES cells, most notably the fixed-tissue macrophages in the liver called Kupffer cells, recognize the C3b/immune complex and break this complex from the RBC by severing the C3b receptor-RBC junction, producing a liberated erythrocyte and a C3b/immune complex which is then engulfed by the Kupffer cells and is completely destroyed within subcellular organelles of the Kupffer cells. This pathogen clearance process, however, is complement-dependent, i.e., confined to immune complexes recognized by the C3b receptor, and is ineffective in removing immune complexes which are not recognized by the C3b receptor.

Taylor et al. have discovered a complement independent method of removing pathogens from the circulatory system. Taylor et al. have shown that chemical crosslinking of a first monoclonal antibody (mAb) specific to a primate C3b receptor to a second monoclonal antibody specific to a pathogenic antigenic molecule creates a bispecific heteropolymeric antibody or bispecific heteropolymer (HP) which offers a mechanism for binding a pathogenic antigenic molecule to a primate's C3b receptor without complement activation (U.S. Pat. Nos. 5,487,890; 5,470,570; and 5,879,679). It was also shown that 7B7, a monoclonal antibody to the bacteriophage ΦX174, was capable of partially neutralizing the bacteriophage when it was cross-linked and presented as an HP, although 7B7 had no neutralizing activity in its monomeric form. Taylor et al., J. of Immunology,158:842-850 (1997). Taylor also reported an HP which can be used to remove a pathogenic antigen specific autoantibody from the circulation. Such an HP, also referred to as an “Antigen-based Heteropolymer” (AHP), contains a CR1 specific monoclonal antibody cross-linked to an antigen (see, e.g., U.S. Pat. No. 5,879,679; Lindorfer et al., 2001, Immunol Rev. 183: 10-24; Lindorfer et al., 2001, J. Immunol Methods 248: 125-138; Ferguson et al., 1995, Arthritis Rheum 38: 190-200).

In addition to HP and AHP produced by cross-linking, bispecific molecules that have a first antigen recognition domain which binds a C3b like receptor, e.g., a complement receptor 1 (CR1), and a second antigen recognition domain which binds an antigen can also be produced by methods that do not involve chemical cross-linking (see, e.g., PCT publication WO 02/46208; and PCT publication WO 01/80883). PCT publication WO 01/80833 describes bispecific antibodies produced by methods involving fusion of hybridoma cell lines, recombinant techniques, and in vitro reconstitution of heavy and light chains obtained from appropriate monoclonal antibodies. PCT publication WO 02/46208 describes bispecific molecules produced by protein trans-splicing.

Developing compositions and methods to reduce infection in animals, e.g., mammals, with pathogens or opportunistic organisms and/or to reduce virulence, e.g., due to toxins, represents a significant challenge.

Current vaccines are impure and chemically complex, eliciting only slow onset of protective immunity, providing incomplete protection, and causing significant adverse reactions. Moreover, the possible use of infectious agents in biological warfare or bioterrorism also demands an improved treatment and/or prevention of anthrax.

Citation or discussion of a reference herein shall not be construed as an admission that such is prior art to the present invention.

### SUMMARY OF THE INVENTION

The present invention provides bispecific molecules comprising an antibody that binds a C3b-like receptor linked with a non-neutralizing antigen-binding antibody that binds an agent, e.g., a pathogenic or opportunistic agent or toxin (e.g., an exotoxin, enterotoxin, or endotoxin) produced by such an agent, including but not limited to, a molecule comprising an epitope of a pathogenic agent. The invention also provides methods of producing the bispecific molecules of the invention as well as methods of therapeutic uses of the bispecific molecules of the invention.

In one aspect, the invention pertains to a bispecific molecule comprising an anti-CR1 antibody linked to a non-neutralizing antibody that binds a pathogenic agent of an animal.

In one embodiment, the non-neutralizing antibody is an enhancing antibody.

In another embodiment, the anti-CR1 antibody is cross-linked to the non-neutralizing antibody that binds the pathogenic agent.

In another embodiment, the pathogenic agent is a bacterium. In another embodiment, the pathogenic agent is a virus. In another embodiment, the pathogenic agent is a microbial toxin.

In another embodiment, at least one of the anti-CR1 antibody and the non-neutralizing antibody are monoclonal antibodies.

In another embodiment, one or more of the antibodies is modified to reduce its immunogenicity. In another embodiment, one or more of the antibodies is deimmunized.

In one embodiment, the first and second antibody are crosslinked using a crosslinking agent. In another embodiment, the crosslinking agent is polyethylene glycol (PEG).

In another embodiment, the anti-CR1 antibody is 7G9. In another embodiment, the anti-CR1 antibody is 19E9.

In another embodiment, the non-neutralizing antibody binds a protective antigen (PA) of a Bacillus anthracis toxin. In another embodiment, the non-neutralizing antibody is 3F3.

In another embodiment, the anti-CR1 antibody is selected from the group consisting of: 7G9 and 19E9.

In one embodiment, the non-neutralizing antibody binds S. aureus. In another embodiment, the non-neutralizing antibody binds protein A.

In another aspect, the invention pertains to a bispecific molecule comprising an anti-CR1 antibody linked to an antibody that is selected from the group consisting of: 3F3, 2F9, 3F10, 3D2, 16E11, 2C11, 6C3, and an antibody that recognizes protein A.

In another aspect, the invention pertains to a bispecific molecule comprising a first antibody that binds a CR1 receptor coupled to a second antibody that binds to a protective antigen component of anthrax toxin but does not inhibit the binding of the protective antigen component of the anthrax toxin to cells.

In another aspect, the invention pertains to a method of treating or preventing a disease associated with presence of a pathogenic agent of an animal in the circulation of a subject, comprising administering to the subject a therapeutically or prophylactically effective amount of a bispecific molecule comprising an anti-CR1 antibody linked to a non-neutralizing antibody that binds to the pathogenic agent.

In one embodiment, the invention pertains to a the non-neutralizing antibody is an enhancing antibody.

In one embodiment, the first and second antibody are crosslinked using a crosslinking agent. In one embodiment, the crosslinking agent is polyethylene glycol (PEG).

In one embodiment, one or more of the antibodies is a monoclonal antibody.

In one embodiment, one or more of the antibodies is modified to reduce its immunogenicity. In one embodiment, the subject is a human.

In one embodiment, the anti-CR1 antibody is selected from the group consisting of: 7G9 and 19E9.

In one aspect, the invention pertains to a method of treating or preventing bacterial infection in a subject, comprising administering to the subject a therapeutically or prophylactically effective amount of a bispecific molecule comprising an anti-CR1 antibody linked to a non-neutralizing antibody that binds to a bacterium.

In one embodiment, the bacterium is a gram negative bacterium.

In one embodiment, the bacterium is a gram positive bacterium. In one embodiment, the bacterium is S. aureus.

In one embodiment, the non-neutralizing antibody is an enhancing antibody.

In one embodiment, the anti-CR1 antibody is cross-linked to the non-neutralizing antibody that binds the bacterium.

In one embodiment, the anti-CR1 antibody and the non-neutralizing antibody are monoclonal antibodies.

In one embodiment, the subject is a human.

In one embodiment, the anti-CR1 antibody is selected from the group consisting of: 7G9 and 19E9.

In one embodiment, the non-neutralizing antibody is an antibody that recognizes protein A.

In one embodiment, the-anti-CR1 antibody is selected from the group consisting of: 7G9 and 19E9.

In one aspect, the invention pertains to a method of treating or preventing a viral infection in an animal subject, comprising administering to the subject a therapeutically or prophylactically effective amount of a bispecific molecule comprising an anti-CR1 antibody linked to a non-neutralizing antibody that binds an epitope of the virus.

In one embodiment, the antibody binds to an envelope (E) protein of the virus.

In one embodiment, the non-neutralizing antibody is an enhancing antibody.

In one embodiment, one or more of the antibodies is a monoclonal antibody.

In one embodiment, the subject is a human

In one embodiment, the anti-CR1 antibody is selected from the group consisting of: 7G9 and 19E9.

In another aspect, the invention pertains to a method of prophylactically preventing or reducing the symptoms of exposure to anthrax spores comprising, administering a bispecific molecule comprising a first antibody that recognizes a C3b receptor coupled to a second antibody that binds to a protective antigen component of anthrax toxin but does not inhibit the binding of the protective antigen component of the anthrax toxin to cells, to a subject at risk of exposure to anthrax spores to thereby prevent or reduce the symptoms of exposure to anthrax spores.

In one embodiment, the C3b receptor is CR1.

In one embodiment, one or more of the antibodies is modified to reduce its immunogenicity.

In one embodiment, one or more of the antibodies is a monoclonal antibody.

In one embodiment, the first and second antibody are crosslinked using a crosslinking agent. In one embodiment, the crosslinking agent is polyethylene glycol (PEG).

In one embodiment, the anthrax toxin is a mutant form that does not bind to antibodies that inhibit the binding of the protective antigen component of the toxin to cells. In one embodiment, the antibody that binds to a protective antigen component of anthrax toxin is selected from the group consisting of: 3F3, 2F9, 3F10, 3D2, 16E11, 2C11 and 6C3.

In another aspect, the invention pertains to a method of reducing the symptoms of exposure to anthrax spores in a population, comprising, administering a bispecific molecule comprising a first antibody that recognizes a C3b receptor coupled to a second antibody that binds to a protective antigen component of anthrax toxin but does not inhibit the binding of the protective antigen component of the anthrax toxin to cells, to multiple subjects at risk of exposure to anthrax spores to thereby prevent or reduce the symptoms of exposure to anthrax spores.

In another aspect, the invention pertains to a method of therapeutically treating the symptoms of exposure to anthrax spores comprising, administering a bispecific molecule comprising a first antibody that recognizes a C3b receptor coupled to a second antibody that binds to a protective antigen component of anthrax toxin but does not inhibit the binding of the protective antigen component of the anthrax toxin to cells, to a subject exposed to anthrax spores to thereby prevent or reduce the symptoms of exposure to anthrax spores.

In one embodiment, the C3b receptor is CR1.

In one embodiment, one or more of the antibodies is modified to reduce its immunogenicity.

In one embodiment, the first and second antibody are crosslinked using a crosslinking agent. In one embodiment, the crosslinking agent is polyethylene glycol (PEG).

In one embodiment, the anthrax toxin is a mutant form that does not bind to antibodies that inhibit the binding of the protective antigen component of the toxin to cells. In one embodiment, the antibody that binds to a protective antigen component of anthrax toxin is selected from the group consisting of: 3F3, 2F9, 3F10, 3D2, 16E11, 2C11 and 6C3.

### BRIEF DESCRIPTION OF THE FIGURES

FIG. 1(A)-(C) shows identification of non-neutralizing anti-PA (protective antigen of B. anthracis) antibodies using macrophage viability assay. Three anti-PA monoclonal antibodies, 3F3, 6C3, and 2F9, showed increased efficiency of delivering PA and lethal factor (LF) of B. anthracis to macrophage and increased efficiency of macrophage killing at certain antibody and/or lethal toxin (containing PA and LF) concentrations. 14B7 was used as a positive control and showed neutralization in all three lethal toxin concentrations. Mouse IgG1 was used as a negative control.

FIG. 2(A)-(B) show that a bispecific molecule, 3F3 cross-linked to 7G9, protected macrophages from the lethal toxin (containing PA and LF) of B. anthracis in the presence of erythrocytes, while 3F3 itself enhanced macrophage killing.

FIG. 3(A)-(B) show that a bispecific molecule, 3F3 cross-linked to 19E9, protected macrophages from the lethal toxin (containing PA and LF) of B. anthracis in the presence of erythrocytes.

FIG. 4(A)-(D) show that a bispecific molecule, 3F3 cross-linked to 7G9, protected macrophages from the lethal toxin (containing PA and LF) of B. anthracis in the presence of soluble CR1.

FIG. 5 shows cytotoxicity of RAW 264.7 macrophages using PA, K684A, and L685A in the presence of Lethal Factor

FIG. 6 shows neutralization of anthrax Lethal toxin (PA+LF), mutant toxins (L685A+LF, K684A+LF) with anti-PA Mab H25.

FIG. 7 shows inactivation of mutant anthrax toxin by HP made using a non-neutralizing Mab 3F3. HP was also made using Mab 14B7 (which is a neutralizing Mab) and is ineffective in inactivating the mutant toxins.

### DETAILED DESCRIPTION OF THE INVENTION

The present invention provides bispecific molecules comprising an antibody that binds a C3b-like receptor linked with a non-neutralizing antigen-binding antibody that binds an agent, e.g., a pathogenic or opportunistic agent, or a toxin produced by such an agent (e.g., an exotoxin, enterotoxin, or endotoxin). Such non-neutralizing antibodies can bind to the pathogenic or opportunistic agent or, e.g., a molecule comprising an epitope of a pathogenic agent. The invention also provides methods of producing the bispecific molecules of the invention as well as methods of therapeutic uses of the bispecific molecules of the invention.

I. Definitions

As used herein, the term “bispecific molecule” includes compounds having two different binding specificities.

As used herein, the term “antibody” includes e.g., naturally occurring antibody or immunoglobulin molecules or genetically engineered antibody molecules that resemble naturally occurring antibody molecules. The term “antibody” as used herein also includes antigen binding fragments of antibody molecules, e.g., fab fragments, scfv molecules, minibodies, and the like.

As used herein, the term “non-neutralizing” with reference to antibody includes antibody molecules or antigen binding fragments that bind to an antigen of a pathogenic agent, in its physiological form (e.g., a form which exists in an animal) but which, used alone, does not prevent or only minimally prevents infection or pathogenic effects of the pathogenic agent. In one embodiment, the non-neutralizing antibody binds to an eptiope of an infectious agent or a toxin which is in a form that is infectious or toxic to cells, e.g., mammalian cells. In one embodiment, the failure to prevent infection or pathogenic effects can be exhibited over the range of practically testable concentrations of the antibody in vivo or in vitro. In another embodiment, minimal prevention of infection or pathogenic effects can be exhibited over the range of practically testable concentrations of the antibody or can be exhibited at low concentrations of the antibody.

A non-neutralizing antibody can, but need not be, an enhancing antibody. The term “enhancing” antibody or fragment thereof to an antigen of a pathogenic agent of an animal refers to an antibody or a fragment thereof that binds to an antigen, in its physiological form, of a pathogenic agent of an animal (preferably a mammal, e.g., primate), and such binding enhances the pathogenic effects of the pathogenic agent at at least some concentrations of the antibody or the pathogenic agent.

In one embodiment, a non-neutralizing antibody is a non-neutralizing anti-PA antibody, wherein the antibody binds to the protective antigen (PA) of B. anthracis (including native PA and recombinantly produced PA), wherein such binding does not prevent the physiological function of PA, i.e., facilitating the entry of the edema factor (EF) and the lethal factor (LF) into cells and causing pathogenic effects. The vegetative B. anthracis bacteria excrete a tripartite exotoxin, which consists of three polypeptides: protective antigen (PA, 83 kDa), lethal factor (LF, 90 kDa) and edema factor (OF, 89 kDa). The two components (OF and LF) of the toxin enzymatically modify substrates within the cytosol of the mammalian cells. OF is an adenylate cyclase that impairs the host defenses through a variety of mechanisms inhibiting phagocytosis and LF is a zinc dependent protease that cleaves several mitogen activated protein kinase kinases (MAPKK) and causes lysis of macrophages. To intoxicate mammalian cells, the third component of the toxin PA, binds to a ubiquitously expressed cellular receptor, Tumor Endothelium Marker-8 (TEM8). In another embodiment, a non-neutralizing antibody can also be a PA enhancing antibody, wherein the antibody binds to PA of B. anthracis and enhances the function of the PA. In another embodiment, a non-neutralizing antibody is a non-neutralizing anti-dengue virus antibody, wherein the antibody binds to an antigenic peptide, e.g., the envelop (E) protein of dengue virus, and such binding does not block the infectivity or injuring effects of a dengue virus. A non-neutralizing antibody or an enhancing antibody can be identified by the macrophage viability assay as described herein.

As used herein the term “pathogen” or “pathogenic agent” includes microorganisms that are capable of infecting or parasitizing normal hosts (e.g., animals (such as mammals, preferably primates, e.g. humans)). As used herein, the term also includes opportunistic agents, e.g., microorganisms that are capable of infecting or parasitizing abnormal hosts, e.g., hosts in which normal flora have been supplanted, e.g., as a result of a treatment regimen, or immunocompromised hosts. As used herein the term also includes microorganisms whose replication is unwanted in a subject or toxic molecules (e.g., toxins) produced by microorganisms.

As used herein, the term crosslinking agent includes agents that participate in protein crosslinking. Crosslinking agents can covalently react with sites on proteins or modified proteins.

II. Bispecific Molecules

A bispecific molecule generally refers to a molecule having two different antigen binding specificities. The bispecific molecules of the present invention comprise an anti-CR1 antibody portion that binds a C3b-like receptor, such as the type 1 complement receptor (CR1 receptor) in primates, and a non-neutralizing antigen-binding antibody portion that binds a pathogenic antigenic molecule, such as but is not limited to an epitope of a pathogen.

As used herein, the term “C3b-like receptor” refers to any mammalian circulatory molecule expressed on the surface of a mammalian blood cell, which has an analogous function to a primate C3b receptor, the CR1, in that it binds to a molecule associated with an immune complex, which is then chaperoned by the blood cell to, e.g., a phagocytic cell for clearance. As used herein, “epitope” refers to an antigenic determinant, i.e., a region of a molecule that provokes an immunological response in a host or is bound by an antibody. This region can but need not comprise consecutive amino acids. The term epitope is also known in the art as “antigenic determinant.” An epitope may comprise as few as three amino acids in a spatial conformation which is unique to the immune system of the host. Generally, an epitope consists of at least five such amino acids, and more usually consists of at least 8-10 such amino acids. Methods for determining the spatial conformation of such amino acids are known in the art.

In the present invention, the anti-CR1 antibody portion and the non-neutralizing antigen-binding antibody portion can be linked by any method known in the art, including but not limited to, cross-linking, fusion of hybridoma cell lines, recombinant techniques, protein trans-splicing, etc.

In the present invention, the anti-CR1 antibody portion of the bispecific molecule can be any antibody that contains a CR1 binding domain and an effector domain. In a preferred embodiment, the anti-CR1 antibody portion is an anti-CR1 monoclonal antibody (mAb). In a preferred embodiment, the anti-CR1 monoclonal antibody is 7G9, HB8592, 3D9, 57F, or 1B4 (see, e.g., Talyor et al., U.S. Pat. No. 5,487,890, which is incorporated herein by reference in its entirety). In another embodiment, the anti-CR1 antibody portion is an anti-CR1 polypeptide antibody, including but is not limited to, a single-chain variable region fragment (scFv) with specificity for a C3b-like receptor fused to the N-terminus of an immunoglobulin Fc domain. The anti-CR1 antibody portion can also be a chimeric antibody in which the complementarity determining regions are mouse, and the framework regions are human thereby decreasing the likelihood of an immune response in human patients treated with the antibody (U.S. Pat. Nos. 4,816,567, 4,816,397, 5,693,762; 5,585,089; 5,565,332 and 5,821,337, each of which is incorporated herein by reference in its entirety). Preferably, the Fc domain of the chimeric antibody can be recognized by the Fc receptors on phagocytic cells, thereby facilitating the transfer and subsequent proteolysis of the immune complex. The anti-CR1 antibody portion can also be a anti-CR1 antibody or antibody fragment that binds a CR1 receptor that has been modified to reduce its immunogenicity in a host (e.g., has been humanized or deimmunized). In some embodiments, the deimmunized anti-CR1 antibody is a deimmunized anti-CR1 monoclonal antibody (mAb). In some embodiments, the constant regions of the deimmunized anti-CR1 antibody are human. In preferred embodiments, the deimmunized anti-CR1 antibody comprises one or more non-human VH or VL sequences modified to comprise one or more amino acid substitutions so that the deimmunized antibody is non-immunogenic or less immunogenic to a human when compared to the respective unmodified non-human sequences (see U.S. Provisional Application No. to be assigned, Attorney Docket No. 9635-039-888, filed on Mar. 28, 2003, which is incorporated herein by reference in its entirety). In a preferred embodiment, the deimmunized anti-CR1 antibody is 19E9, 12H10, 15A12, 44H1, or 31C11. Although, for simplicity, this disclosure often makes references to an anti-CR1 antibody, it will be understood by a skilled artisan that the disclosure is equally applicable to antibodies that binds other C3b-like receptors.

In the present invention, the non-neutralizing antigen-binding antibody portion of the bispecific molecule can be any antigen binding antibody which recognizes and binds an antigenic molecule of a pathogen but which, alone, does not prevent the infection. In specific embodiments, the non-neutralizing antigen-binding antibody is an enhancing antigen-binding antibody, wherein the binding of the antibody to the antigen enhances the pathogenic effects of the pathogen. The non-neutralizing antibody can be a non-neutralizing antibody known in the art. The non-neutralizing antibody can be an antibody that is non-neutralizing determined using in vitro or in vivo testing, for example, the Macrophage Viability Assay described in the Examples. In a specific embodiments, the non-neutralizing antibody is a non-neutralizing anti-PA antibody. In specific embodiments, the non-neutralizing antibody is an enhancing PA-binding antibody, including but not limited to, 3F3, 2F9, 3F10, 3D2, 16E11, 2C11, and 6C3 (see Little et al., Infection and Immunity 56:1807-1813 (1988)). In specific embodiments, the non-neutralizing antibody is a non-neutralizing antibody that binds to dengue virus, including but not limited to, 1A5D, 4A5C, 2B3A, 9A4D, 1B4C (Roehrig et al., Virology 246:317-328 (1998)).

In specific embodiments, the non-neutralizing antibody is an antigen-binding antibody fragment. Preferably, the antigen-binding antibody fragment does not comprise an Fc domain. In a preferred embodiment, the antigen-binding antibody fragment is an Fab, an Fab′, an (Fab′)2, or an Fv fragment of an immunoglobulin molecule. Such an Fab, Fab′ or Fv fragment can be obtained, e.g., from a full antibody by enzymatic processing or from a phage display library by affinity screening and subsequent recombinant expressing (see, e.g., Watkins et al., Vox Sanguinis 78:72-79; U.S. Pat. Nos. 5,223,409 and 5,514,548; PCT Publication No. WO 92/18619; PCT Publication No. WO 91/17271; PCT Publication No. WO 92/20791; PCT Publication No. WO 92/15679; PCT Publication No. WO 93/01288; PCT Publication No. WO 92/01047; PCT Publication No. WO 92/09690; PCT Publication No. WO 90/02809; Fuchs et al., 1991, Bio/Technology 9:1370-1372; Hay et al., 1992, Hum. Antibod. Hybridomas 3:81-85; Huse et al., 1989, Science 246:1275-1281; Griffiths et al., 1993, EMBO J. 12:725-734; and McCafferty et al., 1990, Nature 348:552 554, each of which is incorporated herein by reference in its entirety). In another preferred embodiment, the antigen-binding antibody fragment is a single chain Fv (scFv) fragment which can be obtained, e.g., from a library of phage-displayed antibody fragments by affinity screening and subsequent recombinant expressing. In still another embodiment, the antigen-binding antibody fragment portion of the bispecific molecule is a single-chain antibody (scAb). As used herein, a single-chain antibody (scAb) includes antibody fragments consisting of an scFv fused with a constant domain, e.g., the constant k domain, of a immunoglobulin molecule. In another embodiment, the antigen-binding antibody fragment portion of the bispecific molecule is a Fab, Fab′, (Fab′)₂, Fv, scFv, or scAb fragment fused with a linker peptide of a desired length comprising a chosen amino acid sequence. In preferred embodiment, the linker peptide consists of 1, 2, 5, 10, or 20

The present invention provides a bispecific molecule comprising an anti-CR1 mAb linked to one or more non-neutralizing antigen-binding antibodies. In specific embodiments, the present invention provides a bispecific molecule comprising an anti-CR1 mAb linked to one or more non-neutralizing anti-PA antibodies. In specific embodiments, the present invention provides a bispecific molecule comprising an anti-CR1 mAb linked to one or more enhancing PA-binding antibodies. In specific embodiment, the present invention provides a bispecific molecule comprising an anti-CR1 mAb linked to one or more non-neutralizing antibodies that bind to an antigenic peptide of dengue virus.

In a preferred embodiment, the bispecific molecule comprises an anti-CR1 mAb cross-linked to one or more non-neutralizing antigen-binding antibodies. In specific embodiments of the invention, the bispecific molecule comprises an anti-CR1 mAb cross-linked to one or more non-neutralizing antigen-binding antibody fragments, such as but not limited to Fab, Fab′, (Fab′)₂, Fv, scFv, or scAb fragments. In specific embodiments, the bispecific molecule comprises an anti-CR1 mAb cross-linked to at least 1, 2, 3, 4, 5 or 6 antigen-binding antibody fragments. Preferably, the antigen-binding antibodies or fragments thereof are attached to the anti-CR1 antibody in such a way that their ability to bind the target antigen is not compromised. In preferred embodiments, the bispecific molecule of the invention binds its target antigenic molecule with an activity (e.g., affinity or avidity) at least 5%, 15%, 25%, 50%, 90% or 99% of that of the non-neutralizing antigen-binding antibody. In another preferred embodiments, the bispecific molecule of the invention binds its target antigenic molecule with an activity at least 5%, 15%, 25%, 50%, 90% or 99% of that of the non-neutralizing antigen-binding antibody not cross-linked with the antibody that binds a C3b-like receptor. In one embodiment, the non-neutralizing antigen-binding antibody is attached at a predetermined site to the anti-CR1 antibody. Preferably, such a predetermined site is selected so that the non-neutralizing antigen-binding antibody's antigen-binding affinity is not comprised. More preferably, such a predetermined site is a site on the surface of the non-neutralizing antigen-binding antibody. In a preferred embodiment, the non-neutralizing antigen-binding antibody is attached to the anti-CR1 antibody via a cysteine residue in the non-neutralizing antigen-binding antibody. In another preferred embodiment, the cysteine via which the non-neutralizing antigen-binding antibody is attached to the anti-CR1 antibody is at the C-terminus of the non-neutralizing antigen-binding antibody.

If more than one non-neutralizing antigen-binding antibody are cross-linked to one anti-CR1 antibody, the antigen-binding antibodies can be the same or different. In embodiments in which the more than one non-neutralizing antigen-binding antibodies are different, such non-neutralizing antigen-binding antibodies can bind the same antigenic molecule. The different non-neutralizing antigen-binding antibodies can also bind different antigenic molecules.

The anti-CR1 antibody, e.g., anti-CR1 mAb, and the non-neutralizing antigen-binding antibody(ies) are preferably conjugated by cross-linking via a cross-linker (cross-linking agent). Any cross-linking chemistry known in art for conjugating proteins can be used in the conjunction with the present invention. In a preferred embodiment of the invention, the anti-CR1 mAb and the non-neutralizing antigen-binding antibody are produced using cross-linking agents sulfosuccinimidyl 4 (N maleimidomethyl) cyclohexane 1 carboxylate (sSMCC) and N-succinimidyl-S-acetyl thioacetate (SATA). In another preferred embodiment of the invention, the anti-CR1 mAb and the non-neutralizing antigen-binding antibody are conjugated via a poly-(ethylene glycol) cross-linker (PEG). In this embodiment, the PEG moiety can have any desired length. For example, the PEG moiety can have a molecular weight in the range of 200 to 20,000 Daltons. Preferably, the PEG moiety has a molecular weight in the range of 500 to 1000 Daltons or in the range of 1000 to 8000 Daltons, more preferably in the range of 3250 to 5000 Daltons, and most preferably about 5000 Daltons. Such a bispecific molecule can be produced using cross-linking agents N-succinimidyl-S-acetyl thioacetate (SATA) and a poly(ethylene glycol)-maleimide, e.g., monomethoxy poly(ethylene glycol)-maleimide (mPEG-MAL) or NHS-poly(ethylene glycol)-maleimide (PEG-MAL). Methods of producing PEG-linked bispecific molecules is described in U.S. Provisional Application No. 60/411,731, filed on Sep. 16, 2002, which is incorporated herein by reference.

In still another preferred embodiment, the non-neutralizing antigen-binding antibody is produced with a free thiol by an appropriate host cell (see, e.g., Carter, U.S. Pat. No. 5,648,237, which is incorporated herein by reference in its entirety), and the bispecific molecule is produced by reacting the free thiol containing antibody fragment with an appropriately derivatized, e.g, sSMCC derivatized, anti-CR1 mAb. An anti-CR1 antibody with a free thiol can also be produced directly, ie., without using a chemical cross-linker, e.g., a maleimide. Thus, in another preferred embodiment, the bispecific molecule comprises a monoclonal anti-CR1 antibody conjugated with a non-neutralizing antigen-binding antibody via a disulfide bond. Such a bispecific molecule can be produced by mixing a non-neutralizing antigen-binding antibody having a free thiol with an anti-CR1 antibody with a free thiol.

In another embodiment, the bispecific molecule comprises an anti-CR1 moiety and a non-neutralizing antigen-binding moiety linked by methods that do not involve chemical cross-linking (see e.g., PCT publication WO 02/46208; and PCT publication WO 01/80883, both of which are incorporated herein by reference in their entirety). PCT publication WO 01/80883 describes bispecific molecules produced by methods involving fusion of hybridoma cell lines, recombinant techniques, and in vitro reconstitution of heavy and light chains obtained from appropriate monoclonal antibodies. PCT publication WO 02/46208 describes bispecific molecules produced by protein trans-splicing.

In specific embodiments, the invention provides a bispecific molecule comprising an antibody that binds a C3b-like receptor linked with a non-neutralizing antigen-binding antibody which binds the protective antigen (PA) of bacillus anthracis (anthrax). In one embodiment, the bispecific molecule comprises the anti-CR1 antibody 7G9 cross-linked to the non-neutralizing anti-PA antibody 3F3. The 3F3 antibody is described, e.g., in Little et al. 1988. (Infection and Immunity 56:1807). In another embodiment, the bispecific molecule comprises the deimmunized anti-CR1 antibody 19E9 cross-linked to the non-neutralizing anti-PA antibody 3F3. In specific embodiments, the invention provides a bispecific molecule comprising an antibody that binds a C3b-like receptor linked with a non-neutralizing antigen-binding antibody which binds an antigenic peptide (e.g., E protein) of a dengue virus.

The invention also provides a polyclonal population of bispecific molecules, each comprising an antibody that binds a C3b-like receptor cross-linked with a different non-neutralizing antigen-binding antibody that binds an antigenic molecule. A polyclonal population of bispecific molecules of the present invention refers broadly to any population comprising a plurality of different bispecific molecules, each of which comprises an antibody that binds a C3b-like receptor cross-linked to a different non-neutralizing antigen-binding antibody that binds a pathogenic antigenic molecule. The population thus comprises a plurality of different bispecific molecules having a plurality of different antigen binding specificities via the different non-neutralizing antibodies. The plurality of different non-neutralizing antibodies can recognize and bind the same epitope on a pathogen. The plurality of different antigen binding specificities can also be directed to a plurality of different epitopes on a pathogen. The plurality of different antigen binding specificities can also be directed to a plurality of variants of a pathogen. The plurality of different antigen binding specificities can further be directed to a plurality of different pathogens. The plurality of different antigen recognition of specificities can further be directed to a plurality of different epitopes on a plurality of different pathogens. The characteristic and function of each member bispecific molecule in the plurality of bispecific molecules in the polyclonal population can be known or unknown. The exact proportion of each member bispecific molecule in the plurality of bispecific molecules in the polyclonal population can also be known or unknown. Preferably, the characteristics and the proportions of at least some member bispecific molecules in the plurality of bispecific molecules in the polyclonal population are known so that if desired, the exact proportions of such members can be adjusted for optimal therapeutic and/or prophylactic efficacy. The polyclonal population of bispecific molecules can comprise bispecific molecules that do not bind the target pathogenic antigenic molecule or pathogenic antigenic molecules. For example, the population of bispecific molecules can be prepared from a hyperimmune serum that contains antibodies that bind antigenic molecules other than those that are on the target pathogens. Preferably, the plurality of bispecific molecules in the polyclonal population constitutes at least 1%, 5%, 10%, 20%, 50% or 80% of the population. More preferably, the plurality of bispecific molecules in the polyclonal population constitutes at least 90% of the population. In one embodiment, the plurality of bispecific molecules in the polyclonal population of bispecific molecules preferably does not comprise any single bispecific molecule which has a proportion exceeding 95%, 80%, or 60% of the plurality. More preferably, the plurality of bispecific molecules in the polyclonal population of bispecific molecules does not comprise any single bispecific molecule which has a proportion exceeding 50% of the plurality. In one embodiment, the plurality of bispecific molecules in the polyclonal population comprises at least 2 different bispecific molecules with different antigen binding specificities. Preferably, the plurality of bispecific molecules in the polyclonal population comprises at least 10 different bispecific molecules with different antigen binding specificities. More preferably, the plurality of bispecific molecules in the polyclonal population comprises at least 100 different bispecific molecules with different antigen binding specificities. The polyclonal population can be a polyclonal population generated from a suitable polyclonal population of antigen recognition portions, such as but is not limited to a polyclonal immunoglobulin preparation.

A. Production of Bispecific Molecules

1. Production of Antibodies

The term “antibody” as used herein refers to immunoglobulin molecules or antigen binding portions thereof. Immunoglobulin molecules are encoded by genes which include the kappa, lambda, alpha, gamma, delta, epsilon and mu constant regions, as well as a myriad of immunoglobulin variable regions. Light chains are classified as either kappa or lambda. Light chains comprise a variable light (V_{L}) and a constant light (C_{L}) domain. Heavy chains are classified as gamma, mu, alpha, delta, or epsilon, which in turn define the immunoglobulin classes IgG, IgM, IgA, IgD and IgE, respectively. Heavy chains comprise variable heavy (V_{H}), constant heavy 1 (C_{H}1), hinge, constant heavy 2 (C_{H}2), and constant heavy 3 (C_{H}3) domains. The IgG heavy chains are further sub-classified based on their sequence variation, and the subclasses are designated IgG1, IgG2, IgG3 and IgG4.

Antibodies can be further broken down into two pairs of a light and heavy domain. The paired V_{L} and V_{H} domains each comprise a series of seven subdomains: framework region 1 (FR1), complementarity determining region 1 (CDR1), framework region 2 (FR2), complementarity determining region 2 (CDR2), framework region 3 (FR3), complementarity determining region 3 (CDR3), framework region 4 (FR4) which constitute the antibody-antigen recognition domain.

A chimeric antibody may be made by splicing the genes from a monoclonal antibody of appropriate antigen specificity together with genes from a second human antibody of appropriate biologic activity. More particularly, the chimeric antibody may be made by splicing the genes encoding the variable regions of an antibody together with the constant region genes from a second antibody molecule. This method is used in generating a humanized monoclonal antibody wherein the complementarity determining regions are mouse, and the framework regions are human thereby decreasing the likelihood of an immune response in human patients treated with the antibody (U.S. Pat. Nos. 4,816,567, 4,816,397, 5,693,762; 5,585,089; 5,565,332 and 5,821,337, each of which is incorporated herein by reference in its entirety).

An antibody suitable for use in the present invention may be obtained from natural sources or produced by hybridoma, recombinant or chemical synthetic methods, including modification of constant region functions by genetic engineering techniques (U.S. Pat. No. 5,624,821). The antibody of the present invention may be of any isotype, but is preferably human IgG1.

An antibody can also be a single chain antibody (scFv) which generally comprises a fusion polypeptide consisting of a variable domain of a light chain fused via a polypeptide linker to the variable domain of a heavy chain.

An anti-CR1 mAb that binds a human C3b receptor can be produced by known methods. In one embodiment, anti-CR1 mAb, preferably an anti-CR1 IgG, can be prepared using standard hybridoma procedure known in the art (see, for example, Kohler and Milstein, 1975, Nature 256:495 497; Hogg et al., 1984, Eur. J. Immunol. 14:236-243; O'Shea et al., 1985, J. Immunol. 134:2580-2587; Schreiber, U.S. Pat. No. 4,672,044). A suitable mouse is immunized with human CR1 which can be purified from human erythrocytes. The spleen cells obtained from the immunized mouse are fused with an immortal mouse myeloma cell line which results in a population of hybridoma cells, including a hybridoma that produces an anti-CR1 antibody. The hybridoma which produces the anti-CR1 antibody is then selected, or ‘cloned’, from the population of hybridomas using conventional techniques such as enzyme linked immunosorbent assays (ELISA). Hybridoma cell lines expressing anti-CR1 mAb can also be obtained from various sources, for example, the murine anti-CR1 mAb that binds human CR1 described in U.S. Pat. No. 4,672,044 is available as hybridoma cell line ATCC HB 8592 from the American Type Culture Collection (ATCC). The obtained hybridoma cells are grown and washed using standard methods known in the art. Anti-CR1 antibodies are then recovered from supernatants.

In other embodiments, nucleic acids encoding the heavy and light chains of an anti-CR1 mAb, preferably an anti-CR1 IgG, are prepared from the hybridoma cell line by standard methods known in the art. As a non-limiting example, cDNAs encoding the heavy and light chains of the anti-CR1 IgG are prepared by priming mRNA using appropriate primers, followed by PCR amplification using appropriate forward and reverse primers. Any commercially available kits for cDNA synthesis can be used. The nucleic acids are used in the construction of expression vector(s). The expression vector(s) are transfected into a suitable host. Non-limiting examples include E. coli, yeast, insect cell, and mammalian systems, such as a Chinese hamster ovary cell line. Antibody production can be induced by standard method known in the art. An anti-CR1 antibody can be prepared by immunizing a suitable subject with human CR1 which can be purified from human erythrocytes. The antibody titer in the immunized subject can be monitored over time by standard techniques, such as with an enzyme linked immunosorbent assay (ELISA) using immobilized polypeptide. If desired, the antibody molecules can be isolated from the mammal (e.g., from the blood) and further purified by well-known techniques, such as protein A chromatography to obtain the IgG fraction.

At an appropriate time after immunization, e.g., when the specific antibody titers are highest, antibody-producing cells can be obtained from the subject and used to prepare monoclonal antibodies by standard techniques, such as the hybridoma technique originally described by Kohler and Milstein (1975, Nature 256:495-497), the human B cell hybridoma technique by Kozbor et al. (1983, Immunol. Today 4:72), the EBV-hybridoma technique by Cole et al. (1985, Monoclonal Antibodies and Cancer Therapy, Alan R. Liss, Inc., pp. 77-96) or trioma techniques. The technology for producing hybridomas is well known (see Current Protocols in Immunology, 1994, John Wiley & Sons, Inc., New York, N.Y.). Hybridoma cells producing a monoclonal antibody of the invention are detected by screening the hybridoma culture supernatants for antibodies that bind the polypeptide of interest, e.g., using a standard ELISA assay.

Monoclonal antibodies are obtained from a population of substantially homogeneous antibodies, i.e., the individual antibodies comprising the population are identical except for possible naturally occurring mutations that may be present in minor amounts. Thus, the modifier “monoclonal” indicates the character of the antibody as not being a mixture of discrete antibodies. For example, the monoclonal antibodies may be made using the hybridoma method first described by Kohler et al., 1975, Nature, 256:495, or may be made by recombinant DNA methods (U.S. Pat. No. 4,816,567). The term “monoclonal antibody” as used herein also indicates that the antibody is an immunoglobulin.

In the hybridoma method of generating monoclonal antibodies, a mouse or other appropriate host animal, such as a hamster, is immunized as hereinabove described to elicit lymphocytes that produce or are capable of producing antibodies that will specifically bind to the protein used for immunization (see, e.g., U.S. Pat. No. 5,914,112, which is incorporated herein by reference in its entirety.) Alternatively, lymphocytes may be immunized in vitro. Lymphocytes are then fused with myeloma cells using a suitable fusing agent, such as polyethylene glycol, to form a hybridoma cell (Goding, Monoclonal Antibodies: Principles and Practice, pp. 59 103, Academic Press, 1986). The hybridoma cells thus prepared are seeded and grown in a suitable culture medium that preferably contains one or more substances that inhibit the growth or survival of the unfused, parental myeloma cells. For example, if the parental myeloma cells lack the enzyme hypoxanthine guanine phosphoribosyl transferase (HGPRT or HPRT), the culture medium for the hybridomas typically will include hypoxanthine, aminopterin, and thymidine (HAT medium), which substances prevent the growth of HGPRT deficient cells.

Preferred myeloma cells are those that fuse efficiently, support stable high level production of antibody by the selected antibody producing cells, and are sensitive to a medium such as HAT medium. Among these, preferred myeloma cell lines are murine myeloma lines, such as those derived from MOPC 21 and MPC 11 mouse tumors available from the Salk Institute Cell Distribution Center, San Diego, Calif. USA, and SP 2 cells available from the American Type Culture Collection, Rockville, Md. USA.

Human myeloma and mouse human heteromyeloma cell lines also have been described for the production of human monoclonal antibodies (Kozbor, 1984, J. Immunol., 133:3001; Brodeur et al., Monoclonal Antibody Production Techniques and Applications, pp. 51 63 (Marcel Dekker, Inc., New York, 1987)). Culture medium in which hybridoma cells are growing is assayed for production of monoclonal antibodies directed against the antigen. Preferably, the binding specificity of monoclonal antibodies produced by hybridoma cells is determined by immunoprecipitation or by an in vitro binding assay, such as radioimmunoassay (RIA) or enzyme linked immuno-absorbent assay (ELISA). The binding affinity of the monoclonal antibody can, for example, be determined by the Scatchard analysis of Munson et al., 1980, Anal. Biochem., 107:220. After hybridoma cells are identified that produce antibodies of the desired specificity, affinity, and/or activity, the clones may be subcloned by limiting dilution procedures and grown by standard methods (Goding, Monoclonal Antibodies: Principles and Practice, pp. 59 103 (Academic Press, 1986)). Suitable culture media for this purpose include, for example, D-MEM or RPMI 1640 medium. In addition, the hybridoma cells may be grown in vivo as ascites tumors in an animal. The monoclonal antibodies secreted by the subclones are suitably separated from the culture medium, ascites fluid, or serum by conventional immunoglobulin purification procedures such as, for example, protein A Sepharose, hydroxylapatite chromatography, gel electrophoresis, dialysis, or affinity chromatography.

Alternative to preparing monoclonal antibody-secreting hybridomas, a monoclonal antibody directed against human CR1 can be identified and isolated by screening a recombinant combinatorial immunoglobulin library (e.g., an antibody phage display library) with human CR1. Kits for generating and screening phage display libraries are commercially available (e.g., Pharmacia Recombinant Phage Antibody System, Catalog No. 27-9400-01; and the Stratagene antigen SurfZAP™ Phage Display Kit, Catalog No. 240612). Additionally, examples of methods and reagents particularly amenable for use in generating and screening antibody display library can be found in, for example, U.S. Pat. Nos. 5,223,409 and 5,514,548; PCT Publication No. WO 30 92/18619; PCT Publication No. WO 91/17271; PCT Publication No. WO 92/20791; PCT Publication No. WO 92/15679; PCT Publication No. WO 93/01288; PCT Publication No. WO 92/01047; PCT Publication No. WO 92/09690; PCT Publication No. WO 90/02809; Fuchs et al., 1991, Bio/Technology 9:1370-1372; Hay et al., 1992, Hum. Antibod. Hybridomas 3:81-85; Huse et al., 1989, Science 246:1275-1281; Griffiths et al., 1993, EMBO J. 12:725-734.

In addition, techniques developed for the production of “chimeric antibodies” (Morrison et al., 1984, Proc. Natl. Acad. Sci., 81, 6851-6855; Neuberger et al., 1984, Nature 312, 604-608; Takeda et al., 1985, Nature, 314, 452-454) by splicing the genes from a mouse antibody molecule of appropriate antigen specificity together with genes from a human antibody molecule of appropriate biological activity can be used. A chimeric antibody is a molecule in which different portions are derived from different animal species, such as those having a variable region derived from a murine mAb and a human immunoglobulin constant region. (See, e.g., Cabilly et al., U.S. Pat. No. 4,816,567; and Boss et al., U.S. Pat. No. 4,816,397, each of which is incorporated herein by reference in its entirety)

Humanized antibodies are antibody molecules from non human species having one or more complementarity determining regions (CDRs) from the non human species and a framework region from a human immunoglobulin molecule. (see e.g., U.S. Pat. No. 5,585,089, which is incorporated herein by reference in its entirety.) Such chimeric and humanized monoclonal antibodies can be produced by recombinant DNA techniques known in the art, for example using methods described in PCT Publication No. WO 87/02671; European Patent Application 184,187; European Patent Application 171,496; European Patent Application 173,494; PCT Publication No. WO 86/01533; U.S. Pat. Nos. 4,816,567 and 5,225,539; European Patent Application 125,023; Better et al., 1988, Science 240:1041-1043; Liu et al., 1987, Proc. Natl. Acad. Sci. USA 84:3439-3443; Liu et al., 1987, J. Immunol. 139:3521-3526; Sun et al., 1987, Proc. Natl. Acad. Sci. USA 84:214-218; Nishimura et al., 1987, Canc. Res. 47:999-1005; Wood et al., 1985, Nature 314:446-449; Shaw et al., 1988, J. Natl. Cancer Inst. 80:1553-1559; Morrison 1985, Science 229:1202-1207; Oi et al., 1986, Bio/Techniques 4:214; Jones et al., 1986, Nature 321:552-525; Verhoeyan et al., 1988, Science 239:1534; and Beidler et al., 1988, J. Immunol. 141:4053-4060.

Complementarity determining region (CDR) grafting is another method of humanizing antibodies. It involves reshaping murine antibodies in order to transfer full antigen specificity and binding affinity to a human framework (Winter et al. U.S. Pat. No. 5,225,539). CDR grafted antibodies have been successfully constructed against various antigens, for example, antibodies against IL 2 receptor as described in Queen et al., 1989 (Proc. Natl. Acad. Sci. USA 86:10029); antibodies against cell surface receptors CAMPATH as described in Riechmann et al. (1988, Nature, 332:323; antibodies against hepatitis B in Cole et al. (1991, Proc. Natl. Acad. Sci. USA 88:2869); as well as against viral antigens respiratory syncitial virus in Tempest et al. (1991, Bio Technology 9:267). CDR grafted antibodies are generated in which the CDRs of the murine monoclonal antibody are grafted into a human antibody. Following grafting, most antibodies benefit from additional amino acid changes in the framework region to maintain affinity, presumably because framework residues are necessary to maintain CDR conformation, and some framework residues have been demonstrated to be part of the antigen binding site. However, in order to preserve the framework region so as not to introduce any antigenic site, the sequence is compared with established germline sequences followed by computer modeling.

A deimmunized antibody that binds a human CR1 receptor can also be used in the present invention. As used herein, the term “deimmunized antibody” refers to an antibody that is of a non-human origin but has been modified, i.e., with one or more amino acid substitutions, so that it is non-immunogenic or less immunogenic to a human when compared to the starting non-human antibody. In preferred embodiments, the deimmunized anti-CR1 antibody comprises one or more non-human V_{H} or V_{L} sequences modified to comprise one or more amino acid substitutions so that the deimmunized antibody is non-immunogenic or less immunogenic to a human when compared to the respective unmodified non-human sequences (see WO 00/34317, WO 98/52976, and U.S. Provisional Application No. 60/458,869 filed on Mar. 28, 2003, all of which are incorporated herein by reference in their entirety). In a preferred embodiment, the deimmunized antibody is 19E9.

Completely human antibodies are particularly desirable for therapeutic treatment of human patients. In one embodiment, fully human antibodies can be made using techniques that are known in the art. For example, fully human antibodies against a specific antigen can be prepared by administering the antigen to a transgenic animal which has been modified to produce such antibodies in response to antigenic challenge, but whose endogenous loci have been disabled. Exemplary techniques that can be used to make antibodies are described in U.S. Pat. Nos. 6,150,584; 6,458,592; 6,420,140.

The human immunoglobulin transgenes harbored by the transgenic mice rearrange during B cell differentiation, and subsequently undergo class switching and somatic mutation. Thus, using such a technique, it is possible to produce therapeutically useful IgG, IgA and IgE antibodies. For an overview of this technology for producing human antibodies, see Lonberg and Huszar (1995, Int. Rev. Immunol. 13:65 93). For a detailed discussion of this technology for producing human antibodies and human monoclonal antibodies and protocols for producing such antibodies, see e.g., U.S. Pat. No. 5,625,126; U.S. Pat. No. 5,633,425; U.S. Pat. No. 5,569,825; U.S. Pat. No. 5,661,016; and U.S. Pat. No. 5,545,806. In addition, companies such as Abgenix, Inc. (Freemont, Calif.; see, for example, U.S. Pat. No. 5,985,615) and Medarex, Inc. (Princeton, N.J.), can be engaged to provide human antibodies directed against human CR1 using technology similar to that described above.

Completely human antibodies which recognize and bind a selected epitope can also be generated using a technique referred to as “guided selection.” In this approach a selected non human monoclonal antibody, e.g., a mouse antibody, is used to guide the selection of a completely human antibody recognizing the same epitope (Jespers et al., 1994, Bio/technology 12:899-903).

A pre-existing anti-CR1 antibody, including but not limited to 7G9, HB8592, 3D9, 57F, 1B4 (see, e.g., Talyor et al., U.S. Pat. No. 5,487,890, which is incorporated herein by reference in its entirety), can also be used. In a preferred embodiment, a hybridoma cell line secreting a high-affinity anti-CR1 monoclonal antibody, e.g., 7G9 (murine IgG2a, kappa), is used to generate a master cell bank (MCB). Preferably, the master cell bank is tested for mouse antibody production, mycoplasma and sterility. The anti-CR1 antibody is then produced and purified from ascites fluid. In another preferred embodiment, the anti-CR1 monoclonal antibody used for the production of the bispecific molecules is produced in vitro (hollow-fiber bioreactor) and purified under cGMP. Other techniques are known in the art.

2. Production of Non-Neutralizing Antigen-Binding Antibodies

The non-neutralizing antigen-binding antibody of the bispecific molecule of the invention can be produced by various methods known in the art, e.g., such as those set forth above. The non-neutralizing antigen-binding antibody can be prepared by immunizing a suitable subject with an antigen as an immunogen and then screened by a method known in the art, or the macrophage viability assay described herein. In one embodiment, an organism which is related to the organism to which a non-neutralizing antibody is desired can be used to generate a non-neutralizing antibody. For example, a different virus from the same family can be used. In another embodiment, the same organism to which a non-neutralizing antibody is desired can be used. In another embodiment, a non-neutralizing antibody can be obtained from a subject that has been infected with an organism to which a non-neutralizing antibody is desired or an organism related to one to which a non-neutralizing antibody is desired. The antibody titer in the immunized subject can be monitored over time by standard techniques, such as with an enzyme linked immunosorbent assay (ELISA) using immobilized polypeptide. If desired, the antibody molecules can be isolated from the mammal (e.g., from the blood) and further purified by well-known techniques, such as protein A chromatography to obtain the IgG fraction. At an appropriate time after immunization, e.g., when the specific antibody titers are highest, antibody-producing cells can be obtained from the subject and used to prepare monoclonal antibodies by standard techniques, such as the hybridoma technique originally described by Kohler and Milstein (1975, Nature 256:495-497), the human B cell hybridoma technique by Kozbor et al. (1983, Immunol. Today 4:72), the EBV-hybridoma technique by Cole et al. (1985, Monoclonal Antibodies and Cancer Therapy, Alan R. Liss, Inc., pp. 77-96) or trioma techniques. The technology for producing hybridomas is well known (see generally Current Protocols in Immunology, 1994, John Wiley & Sons, Inc., New York, N.Y.). Hybridoma cells producing a monoclonal antibody of the invention are detected by screening the hybridoma culture supernatants for antibodies that bind the polypeptide of interest, e.g., using a standard ELISA assay.

Monoclonal antibodies are obtained from a population of substantially homogeneous antibodies, i.e., the individual antibodies comprising the population are identical except for possible naturally occurring mutations that may be present in minor amounts. For example, the monoclonal antibodies may be made using the hybridoma method first described by Kohler et al., 1975, Nature, 256:495, or may be made by recombinant DNA methods (e.g., U.S. Pat. No. 4,816,567).

In the hybridoma method of generating monoclonal antibodies, a mouse or other appropriate host animals, such as a hamster, is immunized as described above to elicit lymphocytes that produce or are capable of producing antibodies that will specifically bind to the protein used for immunization (see U.S. Pat. No. 5,914,112, which is incorporated herein by reference in its entirety.)

Alternatively, lymphocytes may be immunized in vitro. Lymphocytes are fused with myeloma cells using a suitable fusing agent, such as polyethylene glycol, to form a hybridoma cell (Goding, Monoclonal Antibodies: Principles and Practice, pp. 59-103 (Academic Press, 1986)). The hybridoma cells thus prepared are seeded and grown in a suitable culture medium that preferably contains one or more substances that inhibit the growth or survival of the unfused, parental myeloma cells. For example, if the parental myeloma cells lack the enzyme hypoxanthine guanine phosphoribosyl transferase (HGPRT or HPRT), the culture medium for the hybridomas typically will include hypoxanthine, aminopterin, and thymidine (HAT medium), which substances prevent the growth of HGPRT-deficient cells.

Preferred myeloma cells are those that fuse efficiently, support stable high-level production of antibody by the selected antibody-producing cells, and are sensitive to a medium such as HAT medium. Among these, preferred myeloma cell lines are murine myeloma lines, such as those derived from MOPC-21 and MPC-11 mouse tumors available from the Salk Institute Cell Distribution Center, San Diego, Calif. USA, and SP-2 cells available from the American Type Culture Collection, Rockville, Md. USA.

Human myeloma and mouse-human heteromyeloma cell lines also have been described for the production of human monoclonal antibodies (Kozbor, 1984, J. Immunol., 133:3001; Brodeur et al., Monoclonal Antibody Production Techniques and Applications, pp. 51-63 (Marcel Dekker, Inc., New York, 1987)). Culture medium in which hybridoma cells are growing is assayed for production of monoclonal antibodies directed against the antigen. Preferably, the binding specificity of monoclonal antibodies produced by hybridoma cells is determined by immunoprecipitation or by an in vitro binding assay, such as radioimmunoassay (RIA) or enzyme-linked immuno-absorbent assay (ELISA). The binding affinity of the monoclonal antibody can, for example, be determined by the Scatchard analysis of Munson et al., 1980, Anal. Biochem., 107:220.

After hybridoma cells are identified that produce antibodies of the desired specificity, affinity, and/or activity, the clones may be subcloned by limiting dilution procedures and grown by standard methods (Goding, Monoclonal Antibodies: Principles and Practice, pp. 59-103 (Academic Press, 1986)). Suitable culture media for this purpose include, for example, D-MEM or RPMI-1640 medium. In addition, the hybridoma cells may be grown in vivo as ascites tumors in an animal. The monoclonal antibodies secreted by the subclones are suitably separated from the culture medium, ascites fluid, or serum by conventional immunoglobulin purification procedures such as, for example, protein A-Sepharose, hydroxylapatite chromatography, gel electrophoresis, dialysis, or affinity chromatography.

Alternative to preparing monoclonal antibody-secreting hybridomas, a monoclonal antibody directed against a pathogen or pathogenic antigenic molecule polypeptide of the invention can be identified and isolated by screening a recombinant combinatorial immunoglobulin library (e.g., an antibody phage display library) with the antigen of interest. Kits for generating and screening phage display libraries are commercially available (e.g., Pharmacia Recombinant Phage Antibody System, Catalog No. 27-9400-01; and the Stratagene antigen SurfZAP™ Phage Display Kit, Catalog No. 240612). Additionally, examples of methods and reagents particularly amenable for use in generating and screening antibody display library can be found in, for example, U.S. Pat. Nos. 5,223,409 and 5,514,548; PCT Publication No. WO 92/18619; PCT Publication No. WO 91/17271; PCT Publication No. WO 92/20791; PCT Publication No. WO 92/15679; PCT Publication No. WO 93/01288; PCT Publication No. WO 92/01047; PCT Publication No. WO 92/09690; PCT Publication No. WO 90/02809; Fuchs et al., 1991, Bio/Technology 9:1370-1372; Hay et al., 1992, Hum. Antibod. Hybridomas 3:81-85; Huse et al., 1989, Science 246:1275-1281; Griffiths et al., 1993, EMBO J. 12:725-734. A phage display library permits selection of desired antibody or antibodies from a very large repertoire of specificities. An additional advantage of a phage display library is that the nucleic acids encoding the selected antibodies can be obtained conveniently, thereby facilitating subsequent construction of expression vectors.

In addition, techniques developed for the production of “chimeric antibodies” (Morrison et al., 1984, Proc. Natl. Acad. Sci., 81, 6851-6855; Neuberger et al., 1984, Nature 312, 604-608; Takeda et al., 1985, Nature, 314, 452-454) by splicing the genes from a mouse antibody molecule of appropriate antigen specificity together with genes from a human antibody molecule of appropriate biological activity can be used. A chimeric antibody is a molecule in which different portions are derived from different animal species, such as those having a variable region derived from a murine mAb and a human immunoglobulin constant region. (See, e.g., Cabilly et al., U.S. Pat. No. 4,816,567; and Boss et al., U.S. Pat. No. 4,816,397, which are incorporated herein by reference in their entirety.)

Humanized antibodies are antibody molecules from non-human species having one or more complementarity determining regions (CDRs) from the non-human species and a framework region from a human immunoglobulin molecule. (see e.g., U.S. Pat. No. 5,585,089, which is incorporated herein by reference in its entirety.) Such chimeric and humanized monoclonal antibodies can be produced by recombinant DNA techniques known in the art, for example using methods described in PCT Publication No. WO 87/02671; European Patent Application 184,187; European Patent Application 171,496; European Patent Application 173,494; PCT Publication No. WO 86/01533; U.S. Pat. Nos. 4,816,567 and 5,225,539; European Patent Application 125,023; Better et al., 1988, Science 240:1041-1043; Liu et al., 1987, Proc. Natl. Acad. Sci. USA 84:3439-3443; Liu et al., 1987, J. Immunol. 139:3521-3526; Sun et al., 1987, Proc. Natl. Acad. Sci. USA 84:214-218; Nishimura et al., 1987, Canc. Res. 47:999-1005; Wood et al., 1985, Nature 314:446-449; Shaw et al., 1988, J. Natl. Cancer Inst. 80:1553-1559; Morrison 1985, Science 229:1202-1207; Oi et al., 1986, Bio/Techniques 4:214; Jones et al., 1986, Nature 321:552-525; Verhoeyan et al., 1988, Science 239:1534; and Beidler et al., 1988, J. Immunol. 141:4053-4060.

Complementarity determining region (CDR) grafting is another method of humanizing antibodies. It involves reshaping murine antibodies in order to transfer full antigen specificity and binding affinity to a human framework (Winter et al. U.S. Pat. No. 5,225,539). CDR-grafted antibodies have been successfully constructed against various antigens, for example, antibodies against IL-2 receptor as described in Queen et al., 1989 (Proc. Natl. Acad. Sci. USA 86:10029); antibodies against cell surface receptors-CAMPATH as described in Riechmann et al. (1988, Nature, 332:323); antibodies against hepatitis B in Cole et al. (1991, Proc. Natl. Acad. Sci. USA 88:2869); as well as against viral antigens-respiratory syncitial virus in Tempest et al. (1991, Bio-Technology 9:267). CDR-grafted antibodies are generated in which the CDRs of the murine monoclonal antibody are grafted into a human antibody. Following grafting, most antibodies benefit from additional amino acid changes in the framework region to maintain affinity, presumably because framework residues are necessary to maintain CDR conformation, and some framework residues have been demonstrated to be part of the antigen binding site. However, in order to preserve the framework region so as not to introduce any antigenic site, the sequence is compared with established germline sequences followed by computer modeling.

Monoclonal antibodies directed against the antigen can be obtained using conventional hybridoma technology. The human immunoglobulin transgenes harbored by the transgenic mice rearrange during B cell differentiation, and subsequently undergo class switching and somatic mutation. Thus, using such a technique, it is possible to produce IgG, IgA and IgE antibodies. For an overview of this technology for producing human antibodies, see Lonberg and Huszar (1995, Int. Rev. Immunol. 13:65-93). For a detailed discussion of this technology for producing human antibodies and human monoclonal antibodies and protocols for producing such antibodies, see e.g., U.S. Pat. No. 5,625,126; U.S. Pat. No. 5,633,425; U.S. Pat. No. 5,569,825; U.S. Pat. No. 5,661,016; and U.S. Pat. No. 5,545,806. In addition, companies such as Abgenix, Inc. (Freemont, Calif. (see, for example, U.S. Pat. No. 5,985,615)) and Medarex, Inc. (Princeton, N.J.), can be engaged to provide human antibodies directed against a selected antigen using technology similar to that described above.

Completely human antibodies which recognize and bind a selected epitope can be generated using a technique referred to as “guided selection.” In this approach a selected non-human monoclonal antibody, e.g., a mouse antibody, is used to guide the selection of a completely human antibody recognizing the same epitope (Jespers et al. (1994) antigen Bio/technology 12:899-903).

A pre-existing antibody directed against a pathogen can be used to isolate additional antigens of the pathogen by standard techniques, such as affinity chromatography or immunoprecipitation for use as immunogens. Moreover, such an antibody can be used to detect the protein (e.g., in a cellular lysate or cell supernatant) in order to evaluate the abundance and pattern of expression of the pathogen. The antibodies can also be used diagnostically to monitor pathogen levels in tissue as part of a clinical testing procedure, e.g., determine the efficacy of a given treatment regimen. The non-neutralizing antigen-binding antibody fragment of the bispecific molecules of the invention can be produced by various methods known in the art.

In one embodiment, the antigen-binding antibody fragment is a fragment of an immunoglobulin molecule containing a binding domain which specifically binds a molecule to be cleared from the circulation of a mammal, e.g., pathogenic antigenic molecule. Examples of immunologically active fragments of immunoglobulin molecules include, but are not limited to, Fab, Fab′ and (Fab′)₂ fragments which can be generated by treating an antibody with an enzyme such as pepsin or papain. In a preferred embodiment, an antigen-binding antibody fragment is produced from a monoclonal antibody having the desired antigen binding specificity. Such a monoclonal antibody can be raised using the targeted antigen by any of the standard methods known in the art. For example, a monoclonal antibody directed against an antigenic molecule can be raised using any one of the methods described, supra, using the antigenic molecule in the place of CR1. The antibody is then treated with pepsin or papain. Pepsin digests an antibody below the disulfide linkages in the hinge region to produce an (Fab′)₂ fragment of the antibody which is a dimer of the Fab composed of a light chain joined to a VH-CH1 by a disulfide bond. The (Fab′)₂ fragments may be reduced under mild conditions to reduce the disulfide linkage in the hinge region thereby converting the (Fab′)₂ dimer to a Fab′ monomer. The Fab′ monomer is essentially an Fab with part of the hinge region. See Paul, ed., 1993, Fundamental Immunology, Third Edition (New York: Raven Press), for a detailed description of epitopes, antibodies and antibody fragments. One of skill in the art will recognize that such Fab′ fragments may be synthesized de novo either chemically or using recombinant DNA technology. Thus, as used herein, the term antibody fragments includes antibody fragments produced by the modification of whole antibodies or those synthesized de novo.

In another embodiment, the method of generating and expressing immunologically active fragments of antibodies described in U.S. Pat. No. 5,648,237, which is incorporated herein by reference in its entirety, is used.

Exemplary methods for producing bispecific molecules comprising antigen binding antibody fragments are disclosed in U.S. Provisional Application No. 60/411,421, filed on Sep. 16 2002, which is incorporated herein by reference in its entirety.

In still another embodiment, the antigen-binding antibody fragment, e.g., an Fv, Fab, Fab′, or (Fab′)₂ is produced by a method comprising affinity screening of a phage display library (see, e.g., Watkins et al., Vox Sanguinis 78:72-79; U.S. Pat. Nos. 5,223,409 and 5,514,548; PCT Publication No. WO 92/18619; PCT Publication No. WO 91/17271; PCT Publication No. WO 92/20791; PCT Publication No. WO 92/15679; PCT Publication No. WO 93/01288; PCT Publication No. WO 92/01047; PCT Publication No. WO 92/09690; PCT Publication No. WO 90/02809; Fuchs et al., 1991, Bio/Technology 9:1370-1372; Hay et al., 1992, Hum. Antibod. Hybridomas 3:81-85; Huse et al., 1989, Science 246:1275-1281; Griffiths et al., 1993, EMBO J. 12:725-734; and McCafferty et al., 1990, Nature 348:552-554, each of which is incorporated herein by reference in its entirety). The nucleic acids encoding the antibody fragment or fragments selected from the phage display library is then obtained for construction of expression vectors. The antibody fragment or fragments can then be produced in a suitable host system, such as a bacterial, yeast, or mammalian host system (see, e.g., Pluckthun et al., Immunotechnology 3:83-105; Adair, Immunological Reviews 130:5-40; Cabilly et al, U.S. Pat. No. 4,816,567; and Carter, U.S. Pat. No. 5,648,237, each of which is incorporated herein by reference in its entirety).

In still another embodiment, techniques described for the production of single chain antibodies (U.S. Pat. No. 4,946,778; Bird, 1988, Science 242:423-426; Huston et al., 1988, Proc. Natl. Acad. Sci. USA 85:5879-5883; and Ward et al., 1989, Nature 334:544-546, each of which is incorporated herein by reference in its entirety) can be adapted to produce single chain antibodies against the antigenic molecule. Single chain antibodies are formed by linking the heavy and light chain fragments of the Fv region via an amino acid bridge, resulting in a single chain polypeptide.

In a preferred embodiment, the non-neutralizing antigen-binding antibody can be modified such that it can be attached at a predetermined site to an anti-CR1 antibody. Preferably, such a predetermined site is selected so that the antigen-binding affinity is not compromised after the fragment is cross-linked to the anti-CR1 antibody. More preferably, such a predetermined site is a site on the surface of the non-neutralizing antigen-binding antibody. In a preferred embodiment, a cysteine residue is engineered into an appropriate location in an non-neutralizing antigen-binding antibody to allow site-specific attachment of the non-neutralizing antigen-binding antibody to an anti-CR1 antibody (see, e.g., Lyons et al., Protein Engineering 3:703-708, which is incorporated herein in its entirety). A skilled person in the art will be able to determine the location where the cysteine residue is introduced as well as the method that can be used to generate such an engineered fragment. In a preferred embodiment, the cysteine is introduced to the C-terminus of the non-neutralizing antigen-binding antibody. In another preferred embodiment, the non-neutralizing antigen-binding antibody containing a cysteine residue is produced by a host cell in such a manner that a cysteinyl free thiol is maintained (see, e.g., Carter, U.S. Pat. No. 5,648,237, which is incorporated herein in its entirety). The non-neutralizing antigen-binding antibody containing cysteinyl free thiol (also referred to as “Ab-fragment-cys-SH”) can then be used to produce the bispecific molecule of the invention directly with an appropriate anti-CR1 antibody or an appropriately derivatized anti-CR1 antibody which can react with the free thiol to form a covalent bond. Anti-CR1 antibody can be a maleimide derivatized anti-CR1 monoclonal antibody, e.g., an anti-CR1 monoclonal antibody derivatized with sulfosuccinimidyl-4-(N maleimidomethyl)-cyclohexane-1-carboxylate (sSMCC) or a poly(ethylene glycol)-maleimide, e.g., monomethoxy poly(ethylene glycol)-maleimide (mPEG-MAL) or NHS-poly(ethylene glycol)-maleimide (PEG-MAL). Alternatively, the anti-CR1 antibody can be a thiolated anti-CR1 antibody, e.g., an anti-CR1 antibody derivatized with N-succinimidyl-S-acetyl thioacetate (SATA), N-succinimidyl-3-(2 pyridyldithio)propionate (SPDP). The Ab-fragment-cys-SH can be cross-linked with the thiolated anti-CR1 antibody via a disulfide bond.

The invention also uses a polyclonal population of non-neutralizing antigen-binding antibodies for production of a polyclonal population of bisepcific molecules. Any method known in the art for producing a polyclonal population of non-neutralizing antigen-binding antibodies can be used in conjunction with the present invention. In preferred embodiments, a population of non-neutralizing antigen-binding antibodies can be produced from a population of antibodies, e.g., a polyclonal population of antibodies, having the desired binding specificities (see, e.g., U.S. Provisional Application No. 60/276,200, filed Mar. 15, 2001; PCT publication WO 02/46208; and PCT publication WO 01/80883, each of which is incorporated herein by reference in its entirety, for methods of producing a polyclonal population of antigen-binding antibodies). In one embodiment, a polyclonal population of antibodies can be produced by immunization of a suitable animal, such as but is not limited to, mouse, rabbit, and horse.

In one embodiment, an immunogenic preparation, typically comprising the antigenic molecules, e.g., associated with the pathogen or pathogens to be cleared from a subject, are used to prepare antibodies by immunizing a suitable subject (e.g., rabbit, goat, mouse or other mammal). An appropriate immunogenic preparation can contain, for example, antigens isolated from cells or tissue sources, antigens recombinantly expressed or antigens chemically synthesized by, e.g., using standard peptide synthesis techniques or attenuated forms of organisms. An immunogenic preparation can also contain chimeric or fusion antigens, which comprise all or part of an antigen for use in the invention, operably linked to a heterologous polypeptide, including but is not limited to a GST fusion antigen in which the antigen is fused to the C-terminus of GST sequences or an immunoglobulin fusion protein in which all or part of an antigen is fused to sequences derived from a member of the immunoglobulin protein family. Chimeric and fusion proteins can be produced by standard recombinant DNA techniques. The preparation can further include an adjuvant, such as Freund's complete or incomplete adjuvant, or similar immunostimulatory agent. A mixture of toxic substances, such as those contained in a reptile or snake bite, can also be used to raise antibody directed to such substances.

The immunogen is then used to immunize a suitable animal. Preferably, the animal is a specialized transgenic animal that can secret human antibody. Non-limiting examples include transgenic mouse strains which can be used to produce a polyclonal population of antibodies directed to a specific pathogen (Fishwild et al., 1996, Nature Biotechnology 14:845-851; Mendez et al., 1997, Nature Genetics 15:146-156). In one embodiment of the invention, transgenic mice that harbor the unrearranged human immunoglobulin genes are immunized with the target immunogens. After a vigorous immune response against the immunogen has been elicited in the mice, the blood of the mice are collected and a purified preparation of human IgG molecules can be produced from the plasma or serum. Any methods known in the art can be used to obtain the purified preparation of human IgG molecules, including but is not limited to affinity column chromatography using anti-human IgG antibodies bound to a suitable column matrix. Anti-human IgG antibodies can be obtained from any sources known in the art, e.g., from commercial sources such as Dako Corporation and ICN. The preparation of IgG molecules produced comprises a polyclonal population of IgG molecules that bind to the immunogen or immunogens at different degree of affinity. Preferably, a substantial fraction of the preparation are IgG molecules specific to the immunogen or immunogens. Although polyclonal preparations of IgG molecules are described, it is understood that polyclonal preparations comprising any one type or any combination of different types of immunoglobulin molecules are also envisioned and are intended to be within the scope of the present invention.

A polyclonal preparation of antibodies or hyperimmune serum directed to a specific pathogen or pathogens and/or pathogenic antigenic molecule or pathogenic antigenic molecules can be produced from human patients who have been infected by the pathogen or pathogens and/or the pathogenic antigenic molecule or pathogenic antigenic molecules using any methods known in the art (see, e.g., Harlow et al., Using Antibodies A Laboratory Manual). As non-limiting examples, hyperimmune serum against parasites, bacteria, and viruses can be prepared according to methods described in, e.g., Shi et al., 1999, American J Tropical Med. Hyg. 60:135-141, Cryz et al., 1986, J. Lab. Clin. Med. 108:182-189, and Cummins et al., 1991, Blood 77:1111-1117. In a preferred embodiment, a polyclonal human IgG preparation is produced using a chromatographic method as described in Tanaka et al., 1998, Brazilian Journal of Medical and Biological Research 31:1375-81, which is incorporated herein by reference in its entirety. Specifically, a combination of ion-exchange, DEAE-Sepharose FF and arginine Sepharose 4B affinity chromatography, and Sephacryl S-300 HR gel filtration is used to produce purified IgG molecules from the gamma-globulin fraction of the human plasma.

However, the present invention is not limited to polyclonal preparations of IgG molecules. It is understood that polyclonal preparations comprising any one type or any combination of different types of immunoglobulin molecules, including but are not limited to IgG, IgE, IgA, etc., are also envisioned and are intended to be within the scope of the present invention. Such polyclonal preparations can be produced using any standard method known in the art. The purified polyclonal preparation is then used in the production of the polyconal population of antigen-binding antibody fragments. A population of antigen-binding antibodies directed to a specific pathogenic antigenic molecule or pathogenic antigenic molecules can be produced from a phage display library. Polyclonal antigen-binding antibody fragments can be obtained by affinity screening of a phage display library having a sufficiently large and diverse population of specificities with an antigen or antigens of interest. Examples of methods and reagents particularly amenable for use in generating and screening antibody display library can be found in, for example, U.S. Pat. Nos. 5,223,409 and 5,514,548; PCT Publication No. WO 92/18619; PCT Publication No. WO 91/17271; PCT Publication No. WO 92/20791; PCT Publication No. WO 92/15679; PCT Publication No. WO 93/01288; PCT Publication No. WO 92/01047; PCT Publication No. WO 92/09690; PCT Publication No. WO 90/02809; Fuchs et al., 1991, Bio/Technology 9:1370-1372; Hay et al., 1992, Hum. Antibod. Hybridomas 3:81-85; Huse et al., 1989, Science 246:1275-1281; Griffiths et al., 1993, EMBO J. 12:725-734; and McCafferty et al., 1990, Nature 348:552 554.

In a preferred embodiment, the polyclonal population of non-neutralizing antigen-binding antibodies directed to a pathogenic antigenic molecule or pathogenic antigenic molecules is produced from a phage display library according to Den et al., 1999, J. Immunol. Meth. 222:45-57; Sharon et al. Comb. Chem. High Throughput Screen. 2000 3:185 96; and Baecher-Allan et al., Comb. Chem. High Throughput Screen. 2000 2:319-325. The phage display library is screened to select a polyclonal sublibrary having binding specificities directed to the antigenic molecule or antigenic molecules of interests by affinity chromatography (McCafferty et al., 1990, Nature 248:552; Breitling et al., 1991, Gene 104:147; and Hawkins et al., 1992, J. Mol. Biol. 226:889). The nucleic acids encoding the heavy and light chain variable regions are then linked head to head to generate a library of bidirectional phage display vectors. The bidirectional phage display vectors are then transferred in mass to bidirectional mammalian expression vectors (Sarantopoulos et al., 1994, J. Immunol. 152:5344) which are used to transfect a suitable hybridoma cell line. The transfected hybridoma cells are induced to produce the antigen-binding antibody fragments using any method known in the art.

In other preferred embodiments, the population of non-neutralizing antigen-binding antibodies directed to a pathogenic antigenic molecule or pathogenic antigenic molecules are produced by a method using the whole collection of selected displayed antibody fragments without clonal isolation of individual members as described in U.S. Pat. No. 6,057,098, which is incorporated by reference herein in its entirety. Polyclonal antigen-binding antibody fragments are obtained by affinity screening of a phage display library having a sufficiently large repertoire of specificities with, e.g., an antigenic molecule having multiple epitopes, preferably after enrichment of displayed library members that display multiple antibodies. The nucleic acids encoding the selected display antibody fragments are excised and amplified using suitable PCR primers. The nucleic acids can be purified by gel electrophoresis such that the full length nucleic acids are isolated. Each of the nucleic acids is then inserted into a suitable expression vector such that a population of expression vectors having different inserts is obtained. The population of expression vectors is then expressed in a suitable host.

3. Production of Bispecific Molecules

A bispecific molecule of the present invention can be a covalent conjugate of one or more non-neutralizing antigen-binding antibodies with an anti-CR1 monoclonal antibody, e.g., the 7G9 antibody as described in U.S. Pat. No. 5,879,679. Any standard chemical cross-linking methods can be used in the present invention. Preferably, a cross-linking method employing a bifunctional cross-linker is used. Preferably, a cross-linking method employing a bifunctional poly(ethylene glycol) cross-linker is used. For example, cross-linking agents, including but not limited to, protein A, glutaraldehyde, carbodiimide, N-succinimidyl S-acetyl thioacetate (SATA), N-succinimidyl-3-(2 pyridyldithio)propionate (SPDP), sulfosuccinimidyl 4-(N maleimidomethyl)-cyclohexane-1-carboxylate (sSMCC), and a poly(ethylene glycol)-maleimide, e.g., monomethoxy poly(ethylene glycol)-maleimide (mPEG-MAL), NHS-poly(ethylene glycol)-maleimide (PEG-MAL), succinimidyl 6-hydrazinonicotinate acetone hydrazone (SANH) or succinimidyl 4-formyl benzoate (SFB) can be used. In a preferred embodiment, SATA is used to derivatize a non-neutralizing antigen-binding antibody. A skilled person in the art will be able to determine the concentrations of the antigen-binding antibody and SATA. In one embodiment, by way of example but not limitation, the following protocol is used. A solution of SATA in DMSO is prepared. The antigen-binding antibody is dialyzed against PBSE buffer. The coupling reaction is initiated by combining the antigen-binding antibody fragment and SATA at a molar ratio of about 1:6. The reactants are mixed by inversion and incubated at room temperature for a desired period of time with mixing. A hydroxylamine HCl solution is prepared by adding hydroxyamine and EDTA to MES. The Hydroxylamine HCl solution is added to the reaction mixture from the SATA coupling step at an appropriate molar ratio, e.g., a molar ratio of about 2000:1, and incubated for a desired period of time at room temperature under argon atmosphere. The reaction mixture is then desalted by chromatography using an Amersham Hi-Prep desalting column in MES buffer. The SATA derivatized antigen-binding antibody can then be used with an appropriately derivatized anti-CR1 antibody, e.g., a maleimide derivatized anti-CR1 antibody, to produce the bispecific molecule of the invention.

In another preferred embodiment, one of the antibodies, e.g., the non-neutralizing antigen-binding antibody containing a cysteine residue is produced by a host cell in such a manner that a free thiol is maintained (see, e.g., Carter, U.S. Pat. No. 5,648,237, which is incorporated herein in its entirety). Preferably, the antigen-binding antibody containing a free thiol is secreted by the host cell. The antigen-binding antibody containing the free thiol can then be recovered and used with an appropriately derivatized anti-CR1 antibody, e.g., a maleimide derivatized anti-CR1 antibody, to produce the bispecific molecule of the invention.

In one embodiment, one of the antibodies, e.g., the anti-CR1 antibody is derivatized with a maleimide using any method known in the art. A skilled person in the art will be able to determine the concentrations of the anti-CR1 antibody and maleimide to achieve a desired number of cross-linking sites on the anti-CR1 antibody. In a preferred embodiment, the antibody is derivatized with maleimide as follows: a fresh stock solution of sSMCC Conjugation solution is prepared in PBSE buffer; the antibody is dialyzed exhaustively against PBSE buffer; the coupling reaction is initiated by combining the antibody and sSMCC at a molar ratio of about 1:6; the reactants are mixed by inversion and incubated at room temperature for 60 min with mixing; and the sSMCC-antibody is recovered by size exclusion chromatography using FPLC with two Pharmacia 26/10 Desalting Columns in series (cat#17-5087-01). The column is preferably pre-washed with distilled water followed by PBSE buffer according to the manufacturer's instructions before loaded with the reaction mixture. The maleimide modified antibody is eluted in the void volume with PBSE buffer and should be used within 15 min. The maleimide derivatized anti-CR1 antibody can then be used with an appropriately antigen-binding antibody fragment, e.g., a SATA derivatized anti-CR1 antibody, to produce the bispecific molecule of the invention.

In another embodiment, one of the antibodies, e.g., the anti-CR1 antibody is derivatized with an poly(ethylene glycol)-maleimide, e.g, NHS-poly(ethylene glycol)-maleimide (PEG-MAL), using any method known in the art. A skilled person in the art will be able to determine the concentrations of the antibody and the PEG-MAL. In this embodiment, the PEG moiety can have any desired length. For example, the PEG moiety can have a molecular weight in the range of 200 to 20,000 Daltons. Preferably, the PEG moiety has a molecular weight in the range of 500 to 1000 Daltons or from 1000 to 8000 Daltons, more preferably in the range of 3250 to 5000 Daltons, and most preferably about 5000 Daltons. Methods of producing PEG-linked bispecific molecules is described in U.S. Provisional Application No. 60/411,731, filed on Sep. 16, 2002. In one embodiment, by way of example but not limitation, the following protocol is used. A MES solution of NHS-PEG-MAL is prepared. The NHS-PEG-MAL solution is added to anti-CR1 antibody, e.g., 7G9, at a molar ratio of about 6:1 (PEG:antibody). The reactants are mixed by inversion and incubated at room temperature for an appropriate period of time with mixing. The reaction mixture is then desalted by chromatography using an Amersham Hi-Prep desalting column in MES buffer. The PEG-maleimide derivatized anti-CR1 antibody can then be used with an appropriately antigen-binding antibody fragment, e.g., a SATA derivatized anti-CR1 antibody, to produce the bispecific molecule of the invention.

In another embodiment, one of the antibodies, e.g., the anti-CR1 antibody is thiolated, e.g., derivatized with N succinimidyl S acetyl thioacetate (SATA), N succinimidyl 3 (2 pyridyldithio)propionate (SPDP). The thiolated anti-CR1 antibody can then be used with an appropriately antigen-binding antibody fragment, e.g., a SATA derivatized anti-CR1 antibody, to produce the bispecific molecule of the invention.

The derivatized antibody, e.g., antibody-maleimide, antibody-PEG-maleimide, or antibody-SH, and the non-neutralizing antigen-binding antibody containing a free thiol, also referred to as Ab-SH, are then combined at a desired molar ratio of derivatized-antibody: non-neutralizing antibody. A skilled person in the art will be able to determine the molar ratio of the derivatized anti-CR1 antibody and non-neutralizing antibody to achieve a desired number of non-neutralizing antigen-binding antibodies to each anti-CR1 antibody. In a preferred embodiment, the maleimide-antibody and Ab-SH are combined at a molar ratio of about 2:1 (derivatized-antibody:Ab-SH). In another preferred embodiment, the derivatized-antibody and antibody-SH are combined at a molar ratio of about 1:1 (derivatized-antibody:Ab-SH). In preferred embodiments, 1, 2, 3, 4, 5 or 6 antigen-binding antibody fragments are conjugated to each anti-CR1 antibody.

In addition, embodiments in which the antigen-binding antibody is derivatized with a maleimide, e.g., sSMCC or NHS-PEG-MAL, whereas the anti-CR1 antibody is, e.g., using SATA or SDPD, are also envisioned.

In a specific embodiment, the method of the invention is used for producing a bispecific molecule comprising an antibody that binds a C3b-like receptor cross-linked with a non-neutralizing antigen-binding antibody which binds the protective antigen (PA) protein of Bacillus anthracis (Anthrax). A non-neutralizing PA-binding antibody is an antibody know in the art (see, e.g., Little et al., 1991, Biochem Biophys Res Commun. 180:531 7; Little et al., 1988, Infect Immun. 56:1807 13), or a PA-binding antibody that is non-neutralizing determined by the assay described herein. In one embodiment, the antibody is 3F3 which binds PA. In a preferred embodiment, the antibody that binds a C3b-like receptor is the murine anti-CR1 IgG 7G9. In a preferred embodiment, the antibody that binds C3b-like receptor is deimmunized anti-CR1 19E9. In a preferred embodiment, the bispecific molecule is produced by cross-linking an anti-CR1 mAb, e.g., 7G9, and a non-neutralizing anti-PA antibody, e.g., 3F3, using N-succinimidyl S-acetyl thioacetate (SATA) and sulfosuccinimidyl 4-(N maleimidomethyl) cyclohexane-1-carboxylate (sSMCC) as the cross-linking agents. In another preferred embodiment, the bispecific molecule is produced by cross-linking an anti-CR1 mAb, e.g, 19E9, and an anti-PA antibody, e.g., 3F3, using N-succinimidyl S-acetyl thioacetate (SATA) and NHS-poly(ethylene glycol)-maleimide (PEG-MAL) as the cross-linking agents.

In another embodiment, a polyclonal population of bispecific molecules of the invention is produced by cross-linking an anti-CR1 antibody described supra and a polyclonal population of antigen-binding antibody fragments described supra, by a method described in this section. See e.g., PCT publication WO 02/46208; and PCT publication WO 01/80883).

In yet another embodiment, the bispecific molecule is produced by a method other than chemical cross-linking, including but not limited to, methods involving fusion of hybridoma cell lines, recombinant techniques, in vitro reconstitution of heavy and light chains obtained from appropriate monoclonal antibodies, and protein trans-splicing. See e.g., PCT publication WO 02/46208 and PCT publication WO 01/80883, all of which are incorporated herein by reference in their entirety.

4. Purification and Testing of Bispecific Molecules

The bispecific molecules produced by a method such as described supra are then preferably purified. Bispecific molecules can be purified by any method known to one skilled in the art using molecular size or specific binding affinity or a combination thereof. In one embodiment, the bispecific molecules can be purified by ion exchange chromatography using columns suitable for isolation of the bispecific molecules of the invention including DEAE, Hydroxylapatite, Calcium Phosphate (see generally Current Protocols in Immunology, 1994, John Wiley & Sons, Inc., New York, N.Y.).

In another embodiment, bispecific molecules are purified by three-step successive affinity chromatography (Corvalan and Smith, 1987, Cancer Immunol. Immunother., 24:127-132): the first column is made of protein A bound to a solid matrix, wherein the Fc portion of the antibody binds protein A, and wherein the antibodies bind the column; followed by a second column that utilizes C3b-like receptor bound to a solid matrix which assays for C3b-like receptor binding via the anti-CR1 mAb portion of the bispecific molecule; and followed by a third column that utilizes specific binding of an antigenic molecule of interest or an antibody which binds the antigen recognition portion of the bispecific molecule.

The bispecific molecules can also be purified by a combination of size exclusion HPLC and affinity chromatography. In one embodiment, the appropriate fraction eluted from size exclusion HPLC is further purified using a column containing a molecule specific to the antigen recognition portion of the bispecific molecule, e.g., an antigenic molecule that can be bound by antigen recognition portion of the bispecific molecule, or an antibody that binds the antigen recognition portion of the bispecific molecule.

The activity of a bispecific molecule, e.g., whether it can inhibit the pathogenic effects of a pathogen, can be tested by a method known in the art, or the macrophage viability assay described infra.

5. Cocktails of Bispecific Molecules

Various purified bispecific molecules can be combined into a “cocktail” of bispecific molecules. Such cocktail of bispecific molecules can include bispecific molecules each having an anti-CR1 mAb conjugated to any one of several desired non-neutralizing antigen-binding antibodies. For example, the bispecific molecule cocktail comprises a plurality of different bispecific molecules, wherein each different bispecific molecule in the plurality contains a different antigen-binding antibody that targets a different pathogens. Such bispecific molecule cocktails are useful as personalized medicine tailored according to the need of individual patients. Alternatively, a cocktail of bispecific molecules can include bispecific molecules each having a different anti-CR1 mAb which binds a different sites on a CR1 receptor conjugated to a desired antigen-binding antibody. Such bispecific molecule cocktails can be used to increase the number of pathogens bound to each red blood cell by utilizing different CR1 binding sites.

6. Ex Vivo Preparation of the Bispecific Molecule

In an alternative embodiment, the bispecific molecule, e.g., 7G9 cross-linked to 3F3, is prebound to hematopoietic cells of the subject ex vivo, prior to administration. For example, hematopoietic cells are collected from the individual to be treated (or alternatively hematopoietic cells from a non-autologous donor of the compatible blood type are collected) and incubated with an appropriate dose of the therapeutic bispecific molecule for a sufficient time so as to allow the antibody to bind the C3b-like receptor on the surface of the hematopoietic cells. The hematopoietic cell/bispecific molecule mixture is then administered to the subject to be treated in an appropriate dose (see, for example, Taylor et al., U.S. Pat. No. 5,487,890). The hematopoietic cells are preferably blood cells, most preferably red blood cells. Accordingly, in a specific embodiment, the invention provides a method of treating a mammal having an undesirable condition associated with the presence of a pathogenic antigenic molecule, comprising the step of administering a hematopoietic cell/bispecific molecule complex to the subject in a therapeutically effective amount, the complex consisting essentially of a hematopoietic cell expressing a C3b-like receptor bound to one or more bispecific molecules. The method alternatively comprises a method of treating a mammal having an undesirable condition associated with the presence of a pathogenic antigenic molecule comprising the steps of (a) contacting a bispecific molecule with hematopoietic cells expressing a C3b-like receptor, to form a hematopoietic cell/bispecific molecule complex; and (b) administering the hematopoietic cell/bispecific molecule complex to the mammal in a therapeutically effective amount.

The invention also provides a method of making a hematopoietic cell/bispecific molecule complex comprising contacting a bispecific molecule with hematopoietic cells that express a C3b-like receptor under conditions conducive to binding, such that a complex forms, the complex consisting essentially of a hematopoietic cell bound to one or more bispecific molecules.

Taylor et al. (U.S. Pat. No. 5,879,679, hereinafter “the '679 patent”) have demonstrated that, in some instances, the system saturates because the concentration of autoantibodies (or other pathogenic antigen) in the plasma is so high that even at the optimum input of bispecific molecules, not all of the autoantibodies can be bound to the hematopoietic cells under standard conditions. For example, for a very high titer of autoantibody sera, a fraction of the autoantibody is not bound to the hematopoietic cells due to its high concentration.

However, saturation can be solved by using combinations of bispecific molecules which contain monoclonal antibodies that bind to different sites on a C3b-like receptor. For example, the monoclonal antibodies 7G9 and 1B4 bind to separate and non competing sites on the primate C3b receptor. Therefore, a “cocktail” containing a mixture of two bispecific molecules, each made with a different monoclonal antibody to the C3b-like receptor, may give rise to greater binding of bispecific molecules to red blood cells. The bispecific molecules of the present invention can also be used in combination with certain fluids used for intravenous infusions.

In yet another embodiment, the bispecific molecule, such as a bispecific molecule, is prebound to red blood cells in vitro as described above, using a blend of at least two different bispecific molecules. In this embodiment, the two different bispecific molecules bind to the same antigen, but also bind to distinct and non overlapping recognition sites on the C3b-like receptor. By using at least two non overlapping bispecific molecules for binding to the C3b-like receptor, the number of bispecific molecule-antigen complexes that can bind to a single red blood cell is increased. Thus, by allowing more than one bispecific molecule to bind to a single C3b-like receptor, antigen clearance is enhanced, particularly in cases where the antigen is in very high concentrations (see for example the '679 patent, column 6, lines 41-64).

III. Characterization of Bispecific Molecules

The bispecific molecules of the invention can be characterized by various methods known in the art. The yield of bispecific molecule can be characterized based on the protein concentration. In one embodiment, the protein concentration is determined using a Lowry assay. Preferably, the bispecific molecule produced by the method of the present invention has a protein concentration of at least 0.100 mg/ml, more preferably at least 2.0 mg/ml, still more preferably at least 5.0 mg/ml, most preferably at least 10.0 mg/ml. In another embodiment, the concentration of the bispecific molecules is determined by measuring UV absorbance. The concentration is determined as the absorbance at 280 nm. Preferably, the bispecific molecule produced by the method of the present invention has an absorbance at 280 nm of at least 0.14.

The bispecific molecule of the invention can also be characterized using any other standard method known in the art. For example, in one embodiment, high performance size exclusion chromatography (HPLC-SEC) assay is used to determine the content of contamination by free IgG proteins. In preferred embodiments, the bispecific molecule composition produced by the method of the present invention has a contaminated IgG concentration of less than 6.0 mg/ml, more preferably less than 2.0 mg/ml, still more preferably less than 0.5 mg/ml, most preferably less than 0.03 mg/ml. In one embodiment, the bispecific molecules can be characterized by using SDS-PAGE to determine the molecular weight of the bispecific molecule.

The bispecific molecule can also be characterized based on the functional activity of the bispecific molecules, e.g., the effectiveness of the molecule in preventing or treating an infection and/or in ameliorating symptoms associated with infection and/or exposure to a toxin can be tested using an in vivo or in vitro model.

For example, in one embodiment, an animal is exposed to, e.g., to a microorganism (e.g., a virus, a bacteria or spore) or a toxin and is treated with an HP with binding specificity for the microorganism or toxin and to CR1. One or more parameters, such as, survival, symptoms, or microbial count (a count of colonies or infectious particles) from the animal can be assessed and compared with that observed in a control animal, an animal not treated with the HP.

In one embodiment, the anti-CR1 binding activity is determined using ELISA with immobilized CR1 receptor molecules (attached to a solid phase, e.g., a microtiter plate) (see Porter et al., U.S. provisional application No. 60/380,211, which is incorporated herein by reference in its entirety). The assay is also referred to as a CR1/Antibody assay or CAA, and can be used generally to measure any anti-CR1 antibody, or HP or AHP containing an anti-CR1 antibody. In a preferred embodiment, ELISA/CR1 plates are prepared by incubating ELISA plates, e.g., high binding flat bottom ELISA plates (Costar EIA/RIA strip plate 2592) with a suitable amount of a bicarbonate solution of CR1 receptors. Preferably, the concentration of the bicarbonate solution of CR1 receptors is 0.2 ug/ml prepared from 5 mg/ml sCR1 receptors stock (Avant Technology Inc.) and a carbonate-bicarbonate buffer (pH 9.6, Sigma C-3041). In a preferred embodiment, 100 ul CR1-bicarbonate solution is dispensed into each well of the ELISA plates and the plates are incubated at 4° C. overnight. The plates are then preferably washed using, e.g., a wash buffer (PBS, 0.1% Tween-20, 0.05% 2-Chloroacetamide). In another preferred embodiment, a SuperBlock Blocking Buffer in PBS (Pierce) is added to the plates for about 30-60 min at room temperature after the wash. The plates can then be dried and stored at 4° C. The titration of anti-CR1 Abs or bispecific molecules can be carried out using a CR1 binding protein, e.g., human anti-CR1 IgG, as the calibrator. In a preferred embodiment, the calibrator a human anti-CR1 IgG having a concentration of 300 or 600 mg/ml. In one embodiment, the titration of the purified composition of bispecific molecules of the invention is carried out using PBS, 0.25% BSA, 0.1% Tween-20 as the diluent buffer, PBS, 0.1% Tween-20, 0.05% 2-Chloroacetamide as the wash buffer, TMB-Liquid Substrate System for ELISA (3,3′, 5.5′-Tetramethyl-Benzidine) and 2N H₂SO₄ as the stop solution. Preferably, the bispecific molecule composition produced by the method of the present invention has an CAA titer of at least 0.10 mg/ml, more preferably at least 0.20 mg/ml, still more preferably at least 0.30 mg/ml, and most preferably at least 0.50 mg/ml. In some embodiments, a specific anti-CR1 activity is determined. The specific anti-CR1 activity is a ratio of CAA titer and protein concentration as determined by lowry or any other protein assay.

The antigen-binding activity can be determined using ELISA with immobilized antigen molecules.

In another embodiment, the bispecificity of a bispecific molecule comprising an antibody that binds a C3b-like receptor cross-linked with a non-neutralizing antigen-binding antibody that binds the protective antigen (PA) protein of Anthrax, i.e., specificities to CR-1 and PA is determined using an ELISA assay. The assay is also referred to as HPCA assay. In a preferred embodiment, ELISA/CR1 plates are prepared as in CAA assay. Calibrators are bispecific molecule 3F3×7G9 (HC=1.0 μg/ml, MC=0.5 μg/ml, LC=0.25 μg/ml). The HPCA assay can be carried out by the following protocol:

A. Binding Bispecific Molecule to CR-1 Plate:

1. Dilute sample bispecific molecule to 5 μg/ml in ELISA diluent (1×PBS buffer, 0.25% BSA, 0.1% Tween 20, 0.05% 2-Chloroacetamide)

2. In a dilution plate, load samples at 5 μg/ml in rows A through H and serially dilute 1:3 fold (a maximum of four samples can be run on one plate). Run all samples, including calibrators in duplicates.

3. Transfer 100 μl of diluted samples from dilution plate into corresponding wells on CR-1 coated plate. Add 100 μl of HC, MC, and LC in duplicates to rows A11 and A12, B11 and B12, C11 and C12, respectively. Add 100 μl of diluent for blanks to five wells in duplicates.

4. Seal plate with the adhesive plate sealer and incubate at 37° C. for 1 hour.

5. Discard solution, wash plate with ELISA wash buffer (1×PBS, 0.1% Tween-20, 0.05% 2-Chloroacetamide) on auto plate washer with 5-cycle program.

B. Binding Biotin Conjugated PA (b-PA) to Bispecific Molecule

1. Dilute b-PA to 5.0 ng/ml in ELISA diluent.

2. Transfer 100 μl of diluted b-PA into all wells (including blank wells).

3. Seal plate with adhesive plate sealer and incubate at 37° C. for 1 hour.

4. Discard solution, wash plate on auto plate washer with 5-cycle program.

C. Binding Horse Radish Peroxidase-Conjugated Streptavidin (SA-HRP, 0.5 mg/ml) to b-PA

1. Dilute SA-HRP 1:10,000 in ELISA diluent.

2. Transfer 100 μl of diluted SA-HRP into all wells (including blank wells).

3. Seal plate with the adhesive plate sealer and incubate at 37° C. for 30 min.

4. Discard solution, wash plate on auto plate washer with 5-cycle program.

D. Signal Development

1. Add 100 μl of pre-warmed TMB (Sigma, cat# T-0440) to all wells.

2. Incubate at room temperature for 15 min (protected from light).

3. Add 100 μl of stop solution (2N H2SO4), incubate at room temperature for 10 min.

4. Read plate at 450 nm using a plate reader.

The maximal absorbance value obtained, referred to as Max OD, can be used as a measure of the total activity of the bispecific molecule. In a preferred embodiment, Max OD is obtained from a 4-parameter sigmoidal fit of the optical density data. In another embodiment, a C50 level is also determined. The C50 is the concentration of a sample which yields 50% of the max OD.

A. Macrophage Viability Assay

The present invention provides a macrophage viability assay system, wherein the survival of macrophages is measured after incubation with one or more molecules. In some embodiments, other types of cell, e.g., erythrocytes, can also be added to the assay system in addition to macrophages. The molecule can be, but is not limited to, a pathogenic agent (including but not limited to, a pathogenic antigen or a toxin), an antigen-binding antibody, an antibody bound to an antigen, a bispecific molecule, a soluble CR1, or a combination thereof. In one embodiment, macrophages are incubated with a pathogenic agent. In another embodiment, macrophages are incubated with both a pathogenic agent and an antibody. In another embodiment, macrophages are incubated with a bispecific molecule and a pathogenic agent. In yet another embodiment, macrophages are incubated with a bispecific molecule, a pathogenic agent, and erythrocytes. Many more combinations are encompassed by the present invention. In specific embodiments, the pathogenic agent is a toxin of B. anthracis (e.g., protective antigen in combination with lethal factor (LF) or in combination with edema factor (EF)). In specific embodiments, the pathogenic agent is a dengue virus. In specific embodiments, the pathogenic antigen is the protective antigen (PA) of B. anthracis. In specific embodiments, the pathogenic antigen is an antigenic peptide (e.g., envelope protein) of dengue virus. In specific embodiments, the antibody is an anti-PA antibody. In specific embodiments, the antibody is an anti-dengue virus antibody. More specifically, the antibody is a non-neutralizing antigen-binding antibody. More specifically, the antibody is an enhancing antibody. In specific embodiments, the bispecific molecule comprises an anti-CR1 antibody linked to an anti-PA antibody. In specific embodiments, the bispecific molecule comprises an anti-CR1 antibody linked to an anti-dengue virus antibody.

The macrophage viability assay can be used for various purposes, e.g., it can be used to determine effects on macrophages by a pathogenic agent or an antibody which binds a pathogenic agent, to screen for non-neutralizing antigen-binding antibodies, or to verify the converting of activities of a non-neutralizing antibody using the bispecific molecule (i.e., HP) system.

In specific embodiments, the present invention provides a method to determine whether an antibody neutralizes or enhances the toxic effect of a pathogenic agent. The method comprises the steps of (1) incubating the antibody with the pathogenic agent under a chosen concentration; (2) adding a known number of macrophages to the incubating mixture and incubate for a period of time; (3) counting the number of dead macrophages, and calculating the percentage of enhancement (% enhancement=100×[(% dead cells with pathogenic agent+Ab)−(% dead cells with pathogenic agent alone)]/(% dead cells with pathogenic agent alone)). In a preferred embodiment, the concentration of the pathogenic agent is chosen such that the survival of the macrophages in the absence of the antibody is at least 30%, at least 40%, at least 50%, at least 60%, at least 70%, at least 80%, or at least 90%. An enhancing antibody can produce a percentage of enhancement that is greater than 20%, greater than 50%, greater than 80%, greater than 100% at some antibody concentration and/or pathogenic agent concentration. In a specific embodiment, the pathogenic agent is the lethal toxin (containing the protective antigen (PA) and the lethal factor (LF)) of B. anthracis, and the antibody is an anti-PA antibody.

In specific embodiments, the present invention provides a method to determine whether an antibody inhibits the toxic effect of a pathogenic agent. The method comprises the steps of (1) incubating the antibody with the pathogenic agent under a chosen concentration; (2) adding a known number of macrophages to the incubating mixture and incubating for a suitable period of time; (3) counting the number of the dead macrophages, and calculating percentage of protection (% protection=100×[(% dead cells with the pathogenic agent alone)−(% dead cells with pathogenic agent+Ab)]/(% dead cells with pathogenic agent alone)). In a preferred embodiment, the concentration of the pathogenic agent is chosen such that the survival of the macrophage in the absence of the antibody is no more than 10%, no more than 20%, no more than 30%, no more than 40%, no more than 50%, no more than 60%, or no more than 70%. A non-neutralizing antibody does not provide protection greater than 5%, greater than 10%, or greater than 20% at some antibody and/or pathogenic agent concentration. An antibody is said to inhibit the toxic effect of a pathogen when the percentage of protection is greater than 0%, greater than 5%, greater than 10%, greater than 20%, greater than 50%, or greater than 80% at some antibody and/or pathogenic agent concentration. In a specific embodiment, the pathogen is the lethal toxin (containing the protective antigen (PA) and the lethal factor (LF)) of B. anthracis, and the antibody is an anti-PA antibody.

In specific embodiments, the present invention provides a method to determine whether a bispecific molecule inhibits the toxic effect of a pathogen. The method comprises the steps of (1) incubating the bispecific molecule with the pathogenic agent; (2) adding erythrocytes or soluble CR1 to the incubation mixture and incubating for a suitable period of time; (3) adding a known number of macrophages to the incubating mixture comprising the erythrocytes and incubating for a suitable period of time; and (4) counting the dead macrophages, and calculating the percentage of protection (% protection=100×[(% dead cells with the pathogenic agent alone)−(% dead cells with pathogenic agent+bispecific molecule)]/(% dead cells with pathogenic agent alone)). A bispecific molecule is said to inhibit the toxic effect of a pathogen when the percentage of protection is greater than 0%, greater than 5%, greater than 10%, or greater than 20% at some antibody and/or pathogen concentration. In a specific embodiment, the pathogen is the lethal toxin (containing the protective antigen (PA) and the lethal factor (LF)) of B. anthracis, and the bispecific molecule comprises an anti-CR1 antibody linked to an anti-PA antibody. The anti-PA antibody can be a non-neutralizing antibody, e.g., an enhancing antibody.

By way of example but not limitation, the procedure for the macrophage viability assay is as follows:

1. Lethal toxin (38.5-150 ng/ml) is added to MAb or HP at various molar ratios of HP or MAb to PA (ratio varying between 2 fold and 0.125 fold of MAb or HP to PA). Duplicates were run for each sample;
2. to one set of samples, 2×10⁸ primate erythrocytes are added where as only medium is added to the other set;
3. after one hour incubation at 37° C., 4×10⁵ J774A.1 cells are added to the above reaction mixture;
4. the cells are incubated with the reaction mixture for 4 hours at 37° C. after which the tubes are washed once with PBS containing 0.5% BSA and 0.1% Sodium-Azide;
5. the cells are stained with a cocktail of anti-CD45 FITC and Propidium Iodide (PI). Excess dye is washed off after a 20 minute incubation at room temperature;
6. the erythrocytes are lysed using BD lysing solution and the cells are washed two times. The cells are then analyzed using flow cytometry done using a BD FACS Calibur; and
7. the CD45 positive population is gated and the dead cell population is the one that is positive for PI staining.
The percent dead cells are determined in each tube and enhancement or protection is calculated as follows:
% enhancement=100×[(% dead cells with LeTx+MAb)−(% dead cells with LeTx alone)]/(% dead cells with LeTx alone); or
% protection=100×[(% dead cells with LeTx alone)−(% dead cells with LeTx+MAb)]/(% dead cells with LeTx alone).
% enhancement=−(% protection).
   IV. Uses of Bispecific Molecules

The bispecific molecules of the present invention are useful in treating or preventing a disease or disorder or any other undesirable condition associated with the presence of a pathogenic antigenic molecule.

The preferred subject for administration of a bispecific molecule of the invention, for therapeutic or prophylactic purposes, is a mammal including but is not limited to non human animals (e.g., horses, cows, pigs, dogs, cats, sheep, goats, mice, rats, etc.), and in a preferred embodiment, is a human or non-human primate. Circulating pathogenic antigenic molecules cleared by the fixed tissue phagocytes include any antigenic moiety that is harmful to the subject. Examples of harmful pathogenic antigenic molecules include any pathogenic antigenic molecule associated with a parasite, fungus, protozoan, bacterium, or virus. Furthermore, circulating pathogenic antigenic molecules may also include toxins, immune complexes, or anything that is present in the circulation and is undesirable or detrimental to the health of the host mammal. Failure of the immune system to effectively remove the pathogenic antigenic molecules from the mammalian circulation can lead to traumatic and hypovolemic shock (Altura and Hershey, 1968, Am. J. Physiol. 215:1414-9).

In specific embodiments, infectious diseases and/or symptoms associated with infection by a microbe are treated or prevented by administration of a bispecific molecule that binds both an antigen of an infectious disease agent and a C3b-like receptor. Thus, in such an embodiment, the pathogenic antigenic molecule is an antigen of an infectious disease agent.

Such antigen can be but is not limited to: influenza virus hemagglutinin (Genbank accession no. J02132; Air, 1981, Proc. Natl. Acad. Sci. USA 78:7639-7643; Newton et al., 1983, Virology 128:495-501), human respiratory syncytial virus G glycoprotein (Genbank accession no. Z33429; Garcia et al., 1994, J. Virol.; Collins et al., 1984, Proc. Natl. Acad. Sci. USA 81:7683), measles virus hemagglutinin (Genbank accession no. M81899; Rota et al., 1992, Virology 188:135-142), herpes simplex virus type 2 glycoprotein gB (Genbank accession no. M 14923; Bzik et al., 1986, Virology 155:322-333), poliovirus I VP1 (Emini et al., 1983, Nature 304:699), envelope glycoproteins of HIV I (Putney et al., 1986, Science 234:1392-1395), hepatitis B surface antigen (Itoh et al., 1986, Nature 308:19; Neurath et al., 1986, Vaccine 4:34), diphtheria toxin (Audibert et al., 1981, Nature 289:543), streptococcus 24M epitope (Beachey, 1985, Adv. Exp. Med. Biol. 185:193), gonococcal pilin (Rothbard and Schoolnik, 1985, Adv. Exp. Med. Biol. 185:247), pseudorabies virus g50 (gpD), pseudorabies virus II (gpB), pseudorabies virus gill (gpC), pseudorabies virus glycoprotein H, pseudorabies virus glycoprotein E, transmissible gastroenteritis glycoprotein 195, transmissible gastroenteritis matrix protein, swine rotavirus glycoprotein 38, swine parvovirus capsid protein, Serpulina hydodysenteriae protective antigen, bovine viral diarrhea glycoprotein 55, Newcastle disease virus hemagglutinin neuraminidase, swine flu hemagglutinin, swine flu neuraminidase, foot and mouth disease virus, hog colera virus, swine influenza virus, African swine fever virus, Mycoplasma hyopneumoniae, infectious bovine rhinotracheitis virus (e.g., infectious bovine rhinotracheitis virus glycoprotein E or glycoprotein G), or infectious laryngotracheitis virus (e.g., infectious laryngotracheitis virus glycoprotein G or glycoprotein I), a glycoprotein of La Crosse virus (Gonzales Scarano et al., 1982, Virology 120:42), neonatal calf diarrhea virus (Matsuno and Inouye, 1983, Infection and Immunity 39:155), Venezuelan equine encephalomyelitis virus (Mathews and Roehrig, 1982, J. Immunol. 129:2763), punta toro virus (Dalrymple et al., 1981, Replication of Negative Strand Viruses, Bishop and Compans (eds.), Elsevier, NY, p. 167), murine leukemia virus (Steeves et al., 1974, J. Virol. 14:187), mouse mammary tumor virus (Massey and Schochetman, 1981, Virology 115:20), hepatitis B virus core protein and/or hepatitis B virus surface antigen or a fragment or derivative thereof (see, e.g., U.K. Patent Publication No. GB 2034323A published Jun. 4, 1980; Ganem and Varmus, 1987, Ann. Rev. Biochem. 56:651 693; Tiollais et al., 1985, Nature 317:489 495), of equine influenza virus or equine herpesvirus (e.g., equine influenza virus type A/Alaska 91 neuraminidase, equine influenza virus type A/Miami 63 neuraminidase, equine influenza virus type A/Kentucky 81 neuraminidase equine herpesvirus type 1 glycoprotein B, and equine herpesvirus type 1 glycoprotein D, antigen of bovine respiratory syncytial virus or bovine parainfluenza virus (e.g., bovine respiratory syncytial virus attachment protein (BRSV G), bovine respiratory syncytial virus fusion protein (BRSV F), bovine respiratory syncytial virus nucleocapsid protein (BRSV N), bovine parainfluenza virus type 3 fusion protein, and the bovine parainfluenza virus type 3 hemagglutinin neuraminidase), bovine viral diarrhea virus glycoprotein 48 or glycoprotein 53. In a preferred embodiment, the antigen is the protective antigen (PA) of B. anthacis.

Additional diseases or disorders that can be treated or prevented by the use of a bispecific molecule of the present invention include, but are not limited to, those caused by hepatitis type A, hepatitis type B, hepatitis type C, influenza, varicella, adenovirus, herpes simplex type I (HSV I), herpes simplex type II (HSV II), rinderpest, rhinovirus, echovirus, rotavirus, respiratory syncytial virus, papilloma virus, papova virus, cytomegalovirus, echinovirus, arbovirus, hantavirus, coxsackie virus, mumps virus, measles virus, rubella virus, polio virus, human immunodeficiency virus type I (HIV I), and human immunodeficiency virus type II (HIV II), any picornaviridae, enteroviruses, caliciviridae, any of the Norwalk group of viruses, togaviruses, such as alphaviruses, flaviviruses, coronaviruses, rabies virus, Marburg viruses, ebola viruses, parainfluenza virus, orthomyxoviruses, bunyaviruses, arenaviruses, reoviruses, rotaviruses, orbiviruses, human T cell leukemia virus type I, human T cell leukemia virus type II, simian immunodeficiency virus, lentiviruses, polyomaviruses, parvoviruses, Epstein Barr virus, human herpesvirus 6, cercopithecine herpes virus 1 (B virus), and poxviruses. In certain embodiments, the virus is not a dengue virus.

Bacterial diseases or disorders that can be treated or prevented by the use of bispecific molecules of the invention include, but are not limited to, Mycobacteria, Rickettsia, Mycoplasma, Neisseria spp. (e.g., Neisseria meningitides and Neisseria gonorrhoeae), Legionella, Vibrio cholerae, Streptococci, such as Streptococcus pneumoniae, Staphylococcus aureus, Staphylococcus epidermidis, Pseudomonas aeruginosa, Corynobacteria diptheriae, Clostridium spp., enterotoxigenic Eschericia coli, and Bacillus anthracis (anthrax), etc. Protozoal diseases or disorders that can be treated or prevented by the use of bispecific molecules of the present invention include, but are not limited to, plasmodia, eimeria, Leishmania, and trypanosoma.

In another embodiment, bispecific molecules of the invention can recognize a toxin produced by a microorganism. Exemplary toxins include, e.g., toxins produced by Bacillus anthracis, Bacillus cereus, Bordatella pertussis, Clostridium botulinum, Clostridium perfringens, Clostridium tetani, Croynebacterium diptheriae, Salmonella sp. Shigella sp., Staphyloccus sp., and Vibrio cholerae.

In a specific embodiment, the invention provides a method and compositions for treating Anthrax infection. The method comprises administrating to a patient a therapeutical sufficient amount of a bispecific molecule comprising an antibody that binds a C3b-like receptor cross-linked with a non-neutralizing antigen-binding antibody which binds the protective antigen (PA) protein of Bacillus anthracis (Anthrax), a common component of the lethal and edema toxins of Anthrax (see, e.g., Little et al., 1991, Biochem Biophys Res Commun. 180:531 7; Little et al., 1988, Infect Immun. 56:1807 13). The protective antigen protein of Anthrax was shown to be required for toxicity (Little et al., 1988, Infect Immun. 56:1807 13). The bispecific molecules can be used to remove PA from the circulation thereby ameliorating the toxic effect of Anthrax. In one embodiment, the non-neutralizing antibody is 3F3 which binds PA (see, e.g., Little et al., 1991, Biochem Biophys Res Commun. 180:531 7; Little et al., 1988, Infect Immun. 56:1807 13). In a preferred embodiment, the antibody that binds a C3b-like receptor is the murine anti-CR1 IgG 7G9. In a preferred embodiment, the antibody that binds a C3b-like receptor is the deimmunized anti-CR1 antibody 19E9. In a preferred embodiment, the bispecific molecule is produced by cross-linking an anti-CR1 mAb, e.g., 7G9, and an anti-PA Fab fragment, e.g., 3F3, using N-succinimidyl-S-acetyl thioacetate (SATA) and sulfosuccinimidyl-4-(N maleimidomethyl) cyclohexane-1-carboxylate (sSMCC) as the cross-linking agents. In another preferred embodiment, the bispecific molecule is produced by cross-linking an anti-CR1 mAb, e.g., 7G9, and a non-neutralizing anti-PA antibody, e.g., 3F3, using N-succinimidyl S-acetyl thioacetate (SATA) and NHS-poly(ethylene glycol)-maleimide (PEG-MAL) as the cross-linking agents. In still another preferred embodiment, the bispecific molecule is produced by cross-linking an anti-CR1 mAb, e.g., 19E9, and a non-neutralizing anti-PA antibody, e.g., 3F3, using N-succinimidyl-S-acetyl thioacetate (SATA) and NHS-poly(ethylene glycol)-maleimide (PEG-MAL) as the cross-linking agents.

In specific embodiments, the present invention provides a method to screen for a non-neutralizing antibody using the macrophage viability assay as described herein. In specific embodiment, the present invention provides a method to screed for an enhancing antibody using the macrophage viability assay as described herein. Such screening is particular useful in preparation of vaccines or other treatment agents that containing an antibody, wherein a non-neutralizing antibody, especially an enhancing antibody, would reduce or impede the therapeutic or prophylactic effects of such vaccine or therapeutic agent.

In specific embodiments, the present invention provides a method to clear a non-neutralizing antibody from the circulation of a subject comprising the steps of (1) identifying a non-neutralizing antibody by macrophage viability assay; (2) raising a second antibody that binds the non-neutralizing antibody; (3) linking the second antibody to an anti-CR1 antibody to construct a bispecific molecule; and (4) administering the bispecific molecule to the subject. In preferred embodiments, the non-neutralizing antibody is an enhancing antibody. In specific embodiments, the non-neutralizing antibody is an enhancing anti-PA antibody or an enhancing anti-dengue virus antibody.

V. Dose of Bispecific Molecules

In one embodiment, the instant invention provides for enhancement of the beneficial or therapeutic activity of an antibody by incorporating that antibody into an HP. In one embodiment, the anti-pathogenic agent antibody component of the HP is a non-neutralizing antibody which, alone, has no neutralizing activity. In another embodiment, the anti-pathogenic agent antibody component of the HP is a non-neutralizing antibody which, alone, has low or minimal neutralizing activity. Incorporation of either such antibody into an HP allows for enhanced beneficial or therapeutic effect of the antibody. As such, in one embodiment, the dose of bispecific molecule administered can be much lower than the dose of antibody alone required to obtain beneficial or therapeutic benefit.

The dose for administration of an HP of the invention can be determined by a physician upon conducting routine tests. Prior to administration to humans, the efficacy is preferably shown in animal models. Any animal model for a blood borne disease known in the art can be used.

More particularly, the dose of the bispecific molecule can be determined based on the hematopoietic cell concentration and the number of C3b-like receptor epitope sites bound by the anti C3b-like receptor monoclonal antibodies per hematopoietic cell. If the bispecific molecule is added in excess, a fraction of the bispecific molecule will not bind to hematopoietic cells, and will inhibit the binding of pathogenic antigens to the hematopoietic cell. The reason is that when the free bispecific molecule is in solution, it will compete for available pathogenic antigen with bispecific molecule bound to hematopoietic cells. Thus, the bispecific molecule mediated binding of the pathogenic antigens to hematopoietic cells follows a bell shaped curve when binding is examined as a function of the concentration of the input bispecific molecule concentration. Viremia may result in up to 108-109 viral particles/ml of blood (HIV is 10⁶/ml; (Ho, 1997, J. Clin. Invest. 99:2565-2567)); the dose of therapeutic bispecific molecules should preferably be, at a minimum, approximately 10 times the antigen number in the blood.

In general, for antibodies, the preferred dosage is 0.01 mg/kg to 10 mg/kg of body weight (generally 0.1 mg/kg to 5 mg/kg). Generally, partially human antibodies and fully human antibodies have a longer half-life within the human body than other antibodies. Accordingly, lower dosages and less frequent administration are often possible. Modifications such as lipidation can be used to stabilize antibodies and to enhance uptake and tissue penetration (e.g., into the brain). A method for lipidation of antibodies is described by Cruikshank et al., 1997, J. Acquired Immune Deficiency Syndromes and Human Retrovirology 14:193.

As defined herein, a therapeutically effective amount of bispecific antibody (i.e., an effective dosage) ranges from about 0.001 to 10 mg/kg body weight, preferably about 0.01 to 5 mg/kg body weight, more preferably about 0.1 to 2 mg/kg body weight, and even more preferably about 0.1 to 1 mg/kg, 0.2 to 1 mg/kg, 0.3 to 1 mg/kg, 0.4 to 1 mg/kg, or 0.5 to 1 mg/kg body weight.

The skilled artisan will appreciate that certain factors may influence the dosage required to effectively treat a subject, including but not limited to the severity of the disease or disorder, previous treatments, the general health and/or age of the subject, and other diseases present. Moreover, treatment of a subject with a therapeutically effective amount of a bispecific antibody can include a single treatment or, preferably, can include a series of treatments. In a preferred example, a subject is treated with a bispecific antibody in the range of between about 0.1 to 5 mg/kg body weight, one time per week for between about 1 to 10 weeks, preferably between 2 to 8 weeks, more preferably between about 3 to 7 weeks, and even more preferably for about 4, 5, or 6 weeks. It will also be appreciated that the effective dosage of a bispecific antibody, used for treatment may increase or decrease over the course of a particular treatment. Changes in dosage may result and become apparent from the results of diagnostic assays as described herein.

It is understood that appropriate doses of bispecific molecule agents depends upon a number of factors within the ken of the ordinarily skilled physician. veterinarian, or researcher. The dose(s) of the bispecific molecule will vary. for example, depending upon the identity, size, and condition of the subject or sample being treated, further depending upon the route by which the composition is to be administered, if applicable, and the effect which the practitioner desires the bispecific molecule to have upon a pathogenic antigenic molecule or autoantibody.

It is also understood that appropriate doses of bispecific molecules depend upon the potency of the bispecific molecule with respect to the antigen to be cleared. Such appropriate doses may be determined using the assays described herein. When one or more of these bispecific molecules is to be administered to an animal (e.g., a human) in order to clear an antigen, a physician, veterinarian, or researcher may, for example, prescribe a relatively low dose at first, subsequently increasing the dose until an appropriate response is obtained. In addition, it is understood that the specific dose level for any particular animal subject will depend upon a variety of factors including the activity of the bispecific molecule employed, the age, body weight, general health, gender, and diet of the subject, the time of administration, the route of administration, the rate of excretion, any drug combination, and the concentration of antigen to be cleared.

VI. Pharmaceutical Formulation and Administration

The bispecific molecules of the invention can be incorporated into pharmaceutical compositions suitable for administration. Such compositions typically comprise bispecific molecule and a pharmaceutically acceptable carrier. As used herein the language “pharmaceutically acceptable carrier” includes, e.g., solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the bispecific molecule, use thereof in the compositions is contemplated. Supplementary bispecific molecules can also be incorporated into the compositions.

A pharmaceutical composition of the invention is formulated to be compatible with its intended route of administration. The preferred route of administration is intravenous. Other examples of routes of administration include parenteral, intradermal, subcutaneous, transdermal (topical), and transmucosal. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersions. For intravenous administration, suitable carriers include physiological saline, bacteriostatic water, Cremophor EL™ (BASF; Parsippany, N.J.) or phosphate buffered saline (PBS). In all cases, the composition must be sterile and should be fluid to the extent that the viscosity is low and the bispecific molecule is injectable. It must be stable under the conditions of manufacture and storage and must be preserved against the contaminating action of microorganisms such as bacteria and fungi.

The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the bispecific molecule (e.g., one or more bispecific molecules) in the required amount in an appropriate solvent with one or a combination of ingredients enumerated above, as required, followed by filtered sterilization. Generally, dispersions are prepared by incorporating the bispecific molecule into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

In one embodiment, the bispecific molecules are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811 which is incorporated herein by reference in its entirety.

It is advantageous to formulate parenteral compositions in dosage unit form for ease of administration and uniformity of dosage. Dosage unit form as used herein refers to physically discrete units suited as unitary dosages for the subject to be treated; each unit containing a predetermined quantity of bispecific molecule calculated to produce the desired therapeutic effect in association with the required pharmaceutical carrier. The specification for the dosage unit forms of the invention are dictated by and directly dependent on the unique characteristics of the bispecific molecule and the particular therapeutic effect to be achieved, and the limitations inherent in the art of compounding such a bispecific molecule for the treatment of individuals. The pharmaceutical compositions can be included in a kit, in a container, pack, or dispenser together with instructions for administration.

VII. Kits

The invention provides kits containing the bispecific molecules, or components necessary to make the bispecific molecules, of the invention. The invention also provides kits containing materials to carry out the macrophage viability assay. The invention also provides kits containing antibodies to a non-neutralizing (including enhancing) antibody, which can be used to screen for such non-neutralizing antibodies. Kits containing the pharmaceutical compositions of the invention are also provided.

### EXAMPLES

The following examples demonstrate that using the bispecific molecule system (i.e., heteropolymer system (HP)), a MAb that by itself does not effectively neutralize the activity of the entity it binds to, can be converted to a reagent that causes destruction of the entity that the MAb binds to. The MAb 3F3 was known to bind to anthrax protective antigen (PA) but does not neutralize toxin activity. When incorporated into an HP in the presence of CR1 or erythrocytes, 3F3 bound to PA and were able to deliver the PA to the macrophage in such a manner as to render the PA incapable of causing cell death. The Examples demonstrated that bispecific molecules (i.e., HPs) of the present invention can therefore be used for treatment of Anthrax infection by removing PA from the circulation. Example 6.1 describes the identification of non-neutralizing anti-PA antibodies using the macrophage viability assay. Example 6.2 describes the in vitro protective effects of a bispecific molecule comprising 3F3 and 7G9. Example 6.3 describes the in vitro protective effects of a bispecific molecule comprising 3F3 and 19E9. Example 6.4 describes the in vitro protective effects of a bispecific molecule comprising 3F3 and 7G9 in the presence of a soluble CR1. For all molar calculations, the molecular weight of a MAb was taken as 150 kDa and the molecular weight of an HP was assumed to be 300 kDa.

### Example 1

### Identifying Non-Neutralizing Anti-PA Antibodies

Macrophage viability assay was used to determine whether an anti-PA antibody is non-neutralizing.

Material and Reagents:

The assay used microtiter well plates with MTT as detection agent. Cells were suspended in DMEM at 10⁶/ml. Macrophage: J774 A1 cells at 6# passage, viability was 93%, passed to 3 plates. Calibration: cell # (×10³): 100, 80, 75, 60, 45, 30, 15, 0. Rest of the wells: 10⁵ cells/well.

Procedure:

1. diluted PA/LF and anti-PA MAbs in a dilution plate;

2. incubated at 37° C. in a CO₂ incubator;

3. transferred 50 μl/well of mix into 100 μl/well macrophage cells;

4. continued incubation at 37° C. CO₂ incubator for 4 hours;

5. added 25 μl/well MTT solution, incubated for 1 hour; and

6. added 100 μl/well lysing/solubilization solution, incubated at 37° C. overnight.

Result:

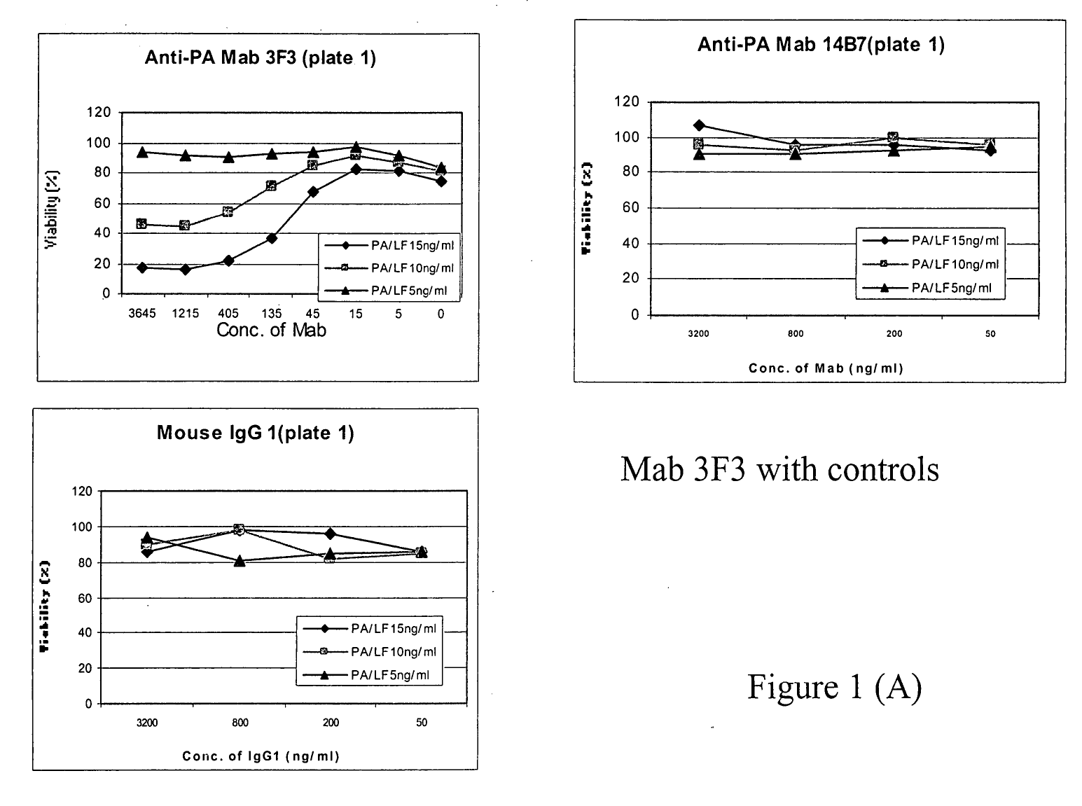

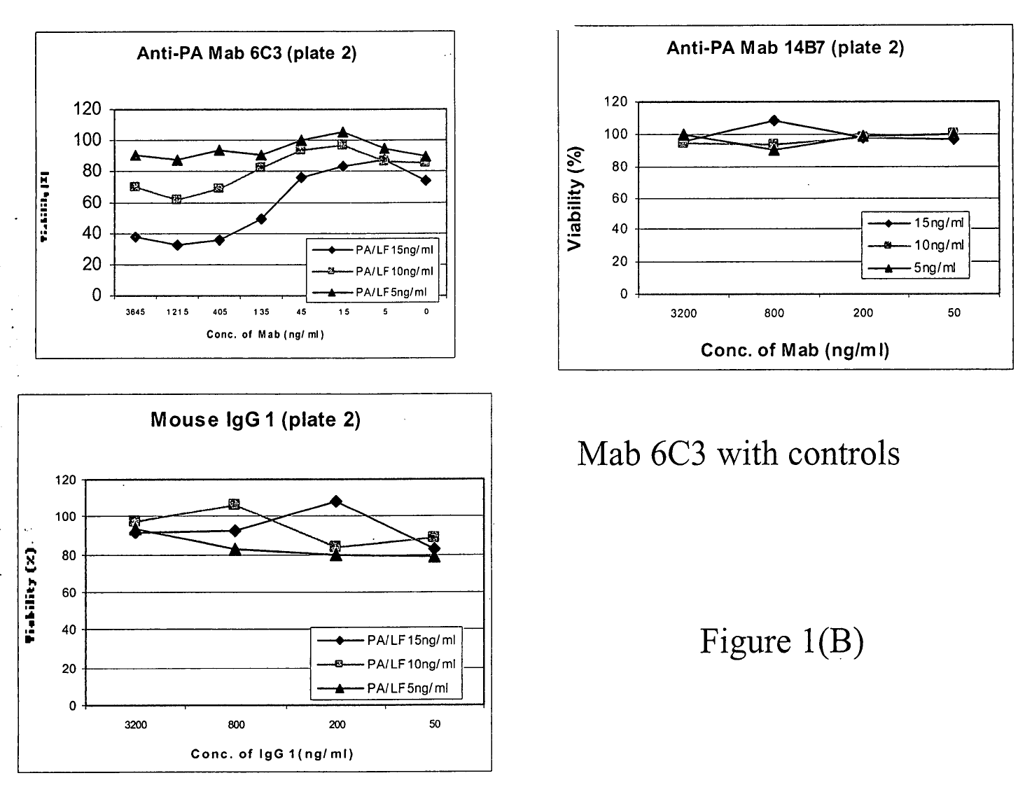

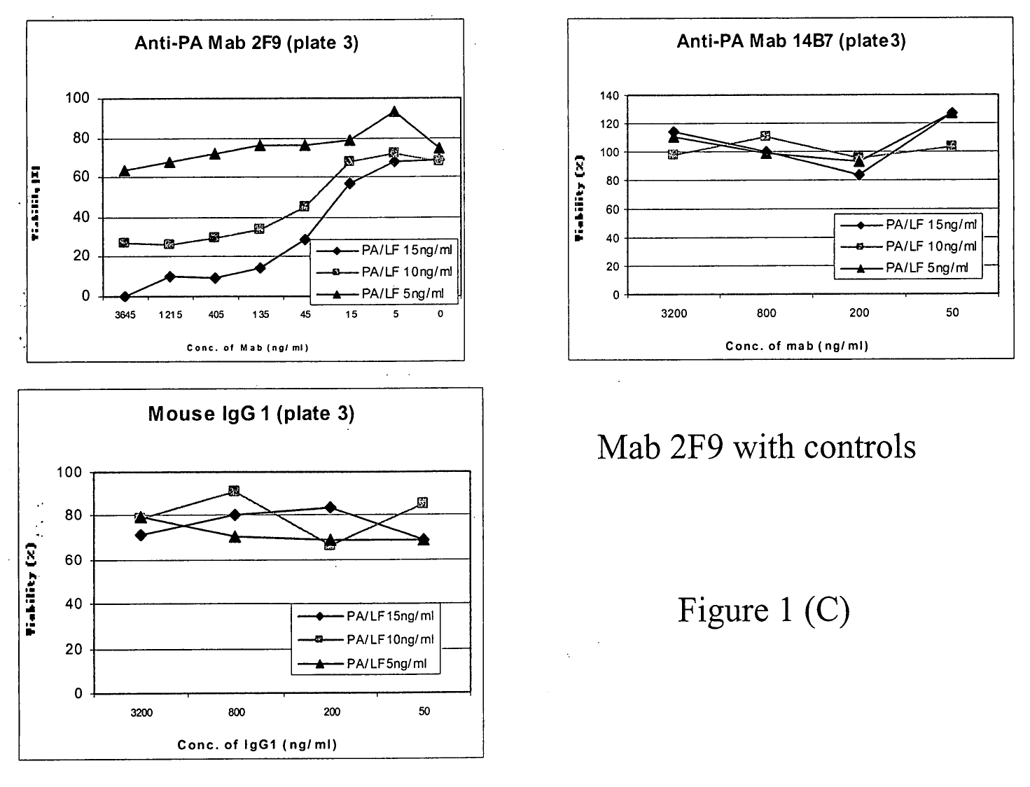

The percentage of survived macrophage cells was plotted against the concentration of the antibodies, and the results are shown in FIG. 1.

Conclusion:

All three anti-PA MAbs showed increased efficiency of delivering PA/LF to macrophage and increased efficiency of macrophage killing in the order of: 2F9>6C3>3F3. The delivery efficiency increased with the concentration of LeTx (lethal toxin, which contains PA and lethal factor (LF)): higher LeTx, more killing. This result showed that the killing of macrophages is dependent on the concentration of LeTx added to the macrophages. 14B7 as the protection positive control showed neutralization in all three LeTx concentrations. Mouse IgG1 as negative control showed some variation.

Bispecific Molecule 3F3/7G9

This experiment is designed to compare the performance of non-neutralizing monoclonal antibody 3F3 and a bispecific molecule comprising 3F3/7G9 in J774 macrophage.

Materials and Reagents:

Monkey Erythrocytes: Macaca fascicularis in Alsevers PPI 1183 pooled, diluted blood to 40% from 100% concentrated (washed) erythrocytes. J774 macrophage cells: passage #5, viability was 88.9%, passed at 2×10⁶ cells/ml. rPA (1.2 mg/ml, 016-01) was diluted 1:100 (495 μl DMEM plus 5 μl PA). Lethal factor (LF) (2.92 mg/ml) was diluted 1:100 (198 μl DMEM plus 2 μl LF). The final concentration of lethal toxin is 38.5 ng/ml. Shaking speed was 2.1.

Samples:

MAb 3F3 was from lot #104-44 (0.78 mg/ml) NM. Actual concentration of MAb 3F3 used in assay was 425.3 μg/ml.

HP 3F3 (bispecific molecule) was from lot #159-45 (970.9 μg/ml). The bispecific molecule comprised 3F3 SATA×7G9 PEG. The bispecific molecule 3F3/7G9 was produced by cross-linking an anti-CR1 MAb, 7G9, and a non-neutralizing anti-PA antibody, 3F3, using N-succinimidyl S-acetyl thioacetate (SATA) and NHS-poly (ethylene glycol)-maleimide (PEG-MAL) as cross-link agents.

Procedure:

1. Diluted HP and MAb as below (based on molar ratio of PA):

TABLE 1

| | | Working | | |
|---|---|---|---|---|
| | Final | stock | | |
| | concen- | concen- | | |
| | tration | tration | | |
| MAb 3F3 | (ng/ml) | (μg/ml) | μl of MAb | cDMEM |
| Mab 1x | 107.6 | 0.86 | 2.03 | 997.97 |
| Mab 0.5x | 53.9 | 0.43 | 400 of 1x | 400 |
| Mab 0.25x | 26.95 | 0.22 | 400 of 0.5x | 400 |
| Mab 0.125x | 13.5 | 0.11 | 400 0f 0.25x | 400 |
| Mab 0.0625X | 6.75 | 0.055 | 400 0f 0.125x | 400 |
| | | Working | | |
| | Final | stock | | |
| | Concen- | concen- | | |
| | tration | tration | | |
| HP 3F3 | (ng/ml) | (μg/ml) | μl of HP | cDMEM |
| HP 1x | 215.6 | 1.72 | 1.77 | 998.23 |
| HP 0.5x | 197.8 | 0.86 | 400 of 1x | 400 |
| HP 0.25x | 53.9 | 0.43 | 400 of 0.5x | 400 |
| HP 0.125x | 27 | 0.22 | 400 of 0.25x | 400 |
| HP 0.0.0625x | 13.5 | 0.11 | 400 0f 0.125x | 400 |

2. diluted lethal toxin and HP or MAb in tubes with Es or medium;

3. PA working stock: the final concentration of rPA (1.2 mg/ml) in cells is 38.5 ng/ml, stock of PA was 0.012 mg/ml (1:100 dilution). The working stock was 8×100 ng/ml (800 ng/ml), add 77 μl of PA stock (12 μg/ml) to 3 ml of cDMEM;

4. LF working stock: the final concentration of LF (2.92 mg/ml) in cells was 34.5 ng/ml, the stock of LF was 29.2 μg/ml, the working stock was 8×100 ng/ml, add 31.5 μl of LF stock (29.2 μg/ml) to 3 ml cDMEM;

5. Add LeTx/HP or LeTx/Mab mixture to 4×10⁵ J774A.1 cells

6. after 3 hour incubation, took cells out from shaker, washed one time with cold PBS with 5% BSA buffer;

7. added 200 μl of the staining solution (containing 15 μl of PI stock, 0.5 μl of anti-CD45-FITC and 184.5 μl of buffer);

8. incubated at 4° C. for 20 minutes, and wash 2 times;

9. added 2 ml of BD FACS lysing solution to all the tubes and incubated at room temperature for 10 minutes;

10. washed 2 times with cold buffer and incubated the final pellet in 400 μl of buffer;

11. analyzed on the FACS calibur within 1 hour.

Results:

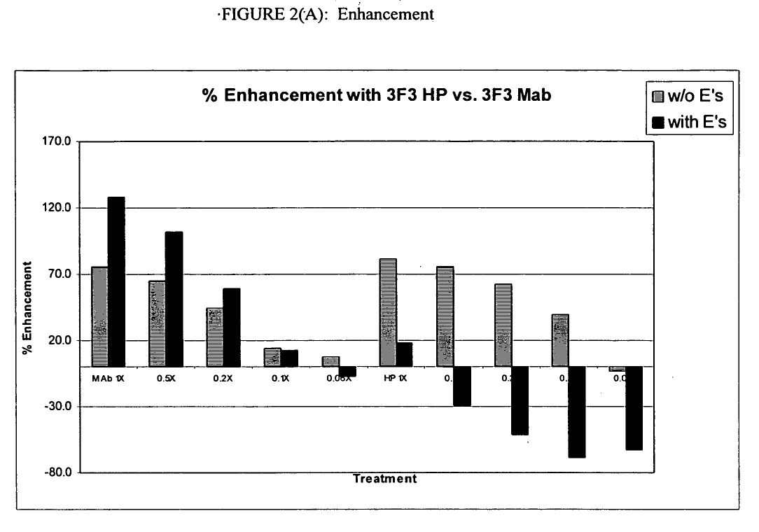

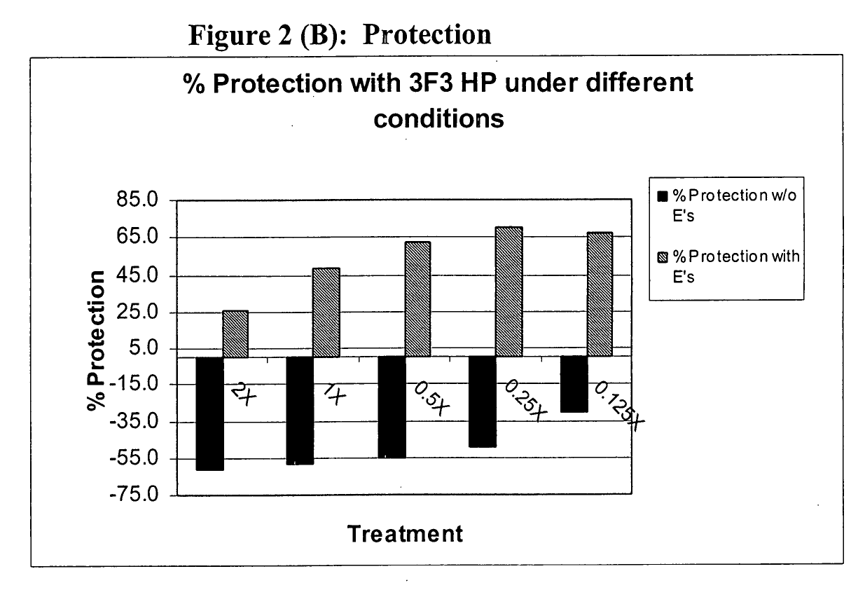

The percentage of enhancement and the percentage of protection by MAb 3F3 and the bispecific molecule 3F3 cross-linked to 7G9 under different conditions are shown in Table 2 and FIG. 2.

TABLE 2

| | Percent dead cells | | | | | | | | | | | |
|---|---|---|---|---|---|---|---|---|---|---|---|---|
| | | | | | | | Mean after | | | | | |
| | | | | | | | background | | | | | |
| | Set 1 | | Set 2 | | Mean Dead | | subtraction | | % Enhancement | | % Protection | |
| | without | | without | | without | | without | | without | | without | |
| | E's | with E's | E's | with E's | E's | with E's | E's | with E's | E's | with E's | E's | with E's |
| Cells only | 1.37 | 1.83 | 2.53 | 1.24 | 1.95 | 1.54 | 0.0 | 0.0 | | | | |
| LeTx | 50.70 | 38.90 | 55.90 | 40.30 | 53.30 | 39.60 | 51.4 | 38.1 | 0.0 | 0.0 | 0.0 | 0.0 |
| MAb 1X | 92.50 | 88.60 | 92.30 | 87.90 | 92.40 | 88.25 | 90.5 | 86.7 | 76.1 | 127.8 | −76.1 | −127.8 |
| 0.5X | 86.50 | 78.10 | 86.60 | 78.50 | 86.55 | 78.30 | 84.6 | 76.8 | 64.8 | 101.7 | −64.8 | −101.7 |
| 0.2X | 76.20 | 59.30 | 76.50 | 65.30 | 76.35 | 62.30 | 74.4 | 60.8 | 44.9 | 59.6 | −44.9 | −59.6 |
| 0.1X | 56.90 | 47.70 | 64.40 | 41.00 | 60.65 | 44.35 | 58.7 | 42.8 | 14.3 | 12.5 | −14.3 | −12.5 |
| 0.06X | 54.10 | 34.60 | 60.50 | 39.40 | 57.30 | 37.00 | 55.4 | 35.5 | 7.8 | −6.8 | −7.8 | 6.8 |
| HP 1X | 94.20 | 44.30 | 95.70 | 49.10 | 94.95 | 46.70 | 93.0 | 45.2 | 81.1 | 18.7 | −81.1 | −18.7 |
| 0.5X | 92.60 | 24.70 | 91.60 | 32.20 | 92.10 | 28.45 | 90.2 | 26.9 | 75.6 | −29.3 | −75.6 | 29.3 |
| 0.2X | 86.30 | 20.60 | 84.90 | 19.70 | 85.60 | 20.15 | 83.7 | 18.6 | 62.9 | −51.1 | −62.9 | 51.1 |
| 0.1X | 72.90 | 15.40 | 74.70 | 11.50 | 73.80 | 13.45 | 71.9 | 11.9 | 39.9 | −68.7 | −39.9 | 68.7 |
| 0.06X | 43.60 | 13.90 | 60.30 | 17.40 | 51.95 | 15.65 | 50.0 | 14.1 | −2.6 | −62.9 | 2.6 | 62.9 |

Conclusion:

The data clearly shows that bispecific molecule 3F3/7G9 (HP) in the presence of Es protect macrophages, while 3F3 itself enhances the killing of the macrophages.

### Example 2

### Comparison of the Performance of Non-Neutralizing Monoclonal Antibody 3F3 and a Bispecific Molecule Comprising 3F3/19E9 in J774 Macrophage

Materials and Reagents:

Monkey Erythrocytes: baboon blood from Lampine Bio Labs, Cat # B1-180N-10, Lot # 102938800 (#4). Macrophage cells: J774A1, passage #3, viability was 94.8%, passed at 2×10⁶ cells/ml. rPA (2.2 mg/ml), Lot # 102-72 (aliquoted by CF) NB199-20, diluted 1:100 (2 μl aliquot+198 μl DMEM). Lethal factor (LF) (1.45 mg/ml), Lot # 199-38. It was diluted 1:100 (2 μl aliquot+198 μl DMEM). Shaking speed was 2.1. HP sample: H4-19E9×3F3 MAb (PEG), Lot # 175-91A, concentration was 309.4 μg/ml. The bispecific molecule was produce by cross-linking a deimmunized anti-CR1 MAb, 19E9, and a non-neutralizing anti-PA antibody, 3F3, using N-succinimidyl S-acetyl thioacetate (SATA) and NHS-poly (ethylene glycol)-maleimide (PEG-MAL) as the cross-linking agents.

Procedure:

1. Diluted HP as below (based on molar ratio of PA): add 50 μl to set with erythrocytes. To the two sets without erythrocytes, add only 25 μl of the HP as described in table below and then add 25 μl of DMEM.

TABLE 3

| | Final | Working stock | | |
|---|---|---|---|---|
| | Concentration | concentration | | |
| HP 3F3 | (ng/ml) | (μg/ml) | μl of HP | DMEM |
| 3x | 1627 | 13.02 | 42.1 | 857.9 |
| 2x | 1664 | 8.67 | 646.7 | 333.36 |
| 1x | 542.2 | 4.34 | 400 of 2x | 400 |
| 0.5x | 271.1 | 2.17 | 400 of 1x | 400 |
| 0.25x | 135.5 | 1.06 | 400 of 0.5x | 400 |
| 0.125x | 67.8 | 0.54 | 400 of 0.25x | 400 |

2. diluted lethal toxin and HP in tubes with Es or with medium;

3. PA working stock concentration: the final concentration of rPA (2.2 mg/ml) in cells was 150.0 ng/ml, stock of PA was 0.022 mg/ml (1:100 dilution). The working stock concentration was 8×150 μg/ml-1.2 μg/ml, added 163.6 til of PA stock (22 μl/ml) to 3 ml of cDMEM;

4. LF working stock concentration: the final concentration of LF (1.45 mg/ml) in cells was 150.0 ng/ml, the stock of LF was 14.5 μg/ml, the working stock concentration was 8×150 ng/ml-1.2 μg/ml, add 245.3 μl of LF stock (14.5 μg/ml) to 3 ml cDMEM;

5. incubated set with erythrocytes with HP for 45 min. in 37° C. incubator. After incubation, washed 1½ time with PBS/BSA;

6. meanwhile, prepared the other set. After 1½ wash for set with erythrocytes, added PA+LF to all tubes at the same time;

7. incubated for 1 hr in 37° C. incubator at a shaking speed of 2.1;

8. added 200 μl of cells at a concentration of 2×10⁶/ml and incubated at 37° C. for 3.5 hrs at a shaking speed of 2.1.

9. after a 3.5 hr incubation, took cells out from the shaker. Washed ½ times with cold PBS/0.5% BSA buffer;

10. added 200 μl of BD FACS lysing solution to all the tubes and incubated at room temperature for 10 min;

11. incubated at 4° C. for 20 min. and washed 1½ times;

12. added 2 ml of BD FACS lysing solution to all the tubes and incubates at room temperature for 10 min.;

13. washed 1½ times with cold buffer and incubated the final pellet in 400 μl of buffer;

14. analyzed on the FACS calibur within 1 hour.

Results:

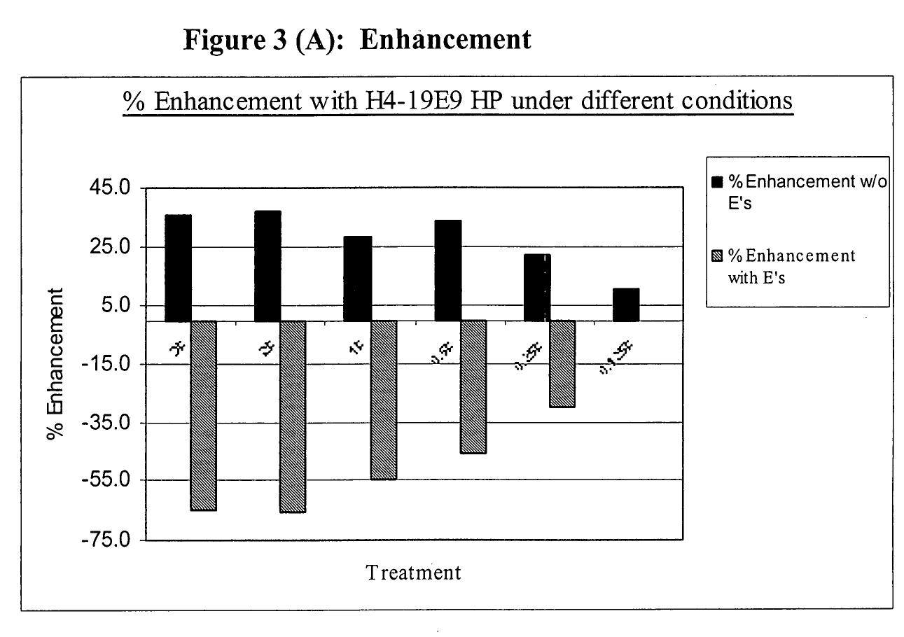

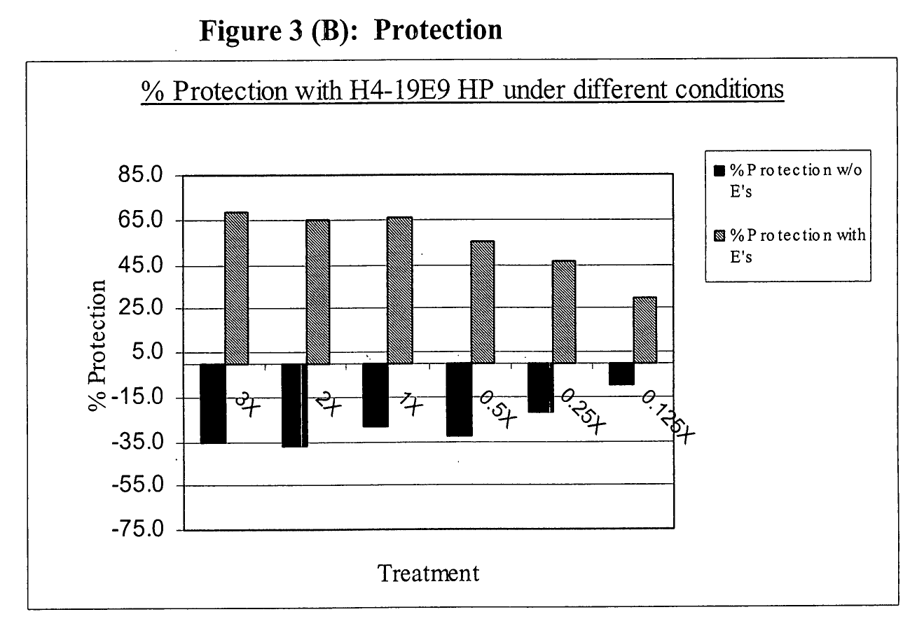

The percentage of enhancement and the percentage of protection of the bispecific molecule 19E9 cross-linked to 3F3 under different conditions are shown in Table 4 and FIG. 3.

TABLE 4

| | | | | | | | Mean w/. | | | | | |
|---|---|---|---|---|---|---|---|---|---|---|---|---|
| | | | | | | | Background | | % | | | |
| | Set 1 | | | | Mean | | subt. | | Enhancement | | % protection | |
| | w/o | with | Set 2 | with | w/o | with | w/o | | w/o | | w/o | |
| | E's | E's | w/o E's | E's | E's | E's | E's | with E's | E's | with E's | E's | with E's |
| Cells | 0.58 | 0.26 | 0.37 | 1.29 | 0.48 | 0.78 | 0.0 | 0.0 | | | | |
| only | | | | | | | | | | | | |
| LeTx | 69.20 | 44.90 | 70.90 | 51.90 | 70.05 | 48.40 | 69.6 | 47.6 | 0.0 | 0.0 | 0.0 | 0.0 |
| 3X | 93.40 | 16.60 | 95.80 | 15.10 | 94.60 | 15.85 | 94.1 | 15.1 | 35.2 | −68.3 | −35.2 | 68.3 |
| 2X | 96.60 | 17.90 | 94.90 | 16.90 | 95.75 | 17.40 | 95.3 | 16.6 | 36.9 | −65.1 | −36.9 | 65.1 |
| 1X | 87.90 | 19.30 | 91.50 | 14.80 | 89.70 | 17.05 | 89.2 | 16.3 | 28.2 | −65.8 | −28.2 | 65.8 |
| 0.5X | | 21.60 | 93.20 | 23.10 | 93.20 | 22.35 | 92.7 | 21.6 | 33.2 | −54.7 | −33.2 | 54.7 |
| 0.25X | | 25.2 | 85.6 | 27.7 | 85.6 | 26.45 | 85.1 | 25.7 | 22.3 | −46.1 | −22.3 | 46.1 |
| 0.125X | | 37.00 | 77.30 | 31.60 | 77.30 | 34.30 | 76.8 | 33.5 | 10.4 | −29.6 | −10.4 | 29.6 |

Conclusion:

The data clearly shows that bispecific molecule 3F3/19E9 (HP) in the presence of Es protect macrophages from the lethal toxin.

### Example 3

### Macrophage Viability Assay with Soluble CR1

Examples 1 and 2 demonstrated that HP in solution behaves similar to the MAb as expected since it was not bound to erythrocytes and was unable to clear PA. However, when HP was used in the presences of erythrocytes, there was protection of the macrophage cells. There could be two possible reasons for the observed protection of macrophages with the HP in the presence of erythrocytes: 1) the PA is physically cleared to the erythrocytes via the HP, thereby preventing the PA to bind its receptor on the cell surface. This subsequently leads to lesser internalization of LF and therefore less killing; or 2) the binding of the 7G9 component of HP 7G9×3F3 to CR1 activates the Fc of the 7G9 and directs the immune complex for destruction via the Fc-mediated pathway.

To test these hypotheses, soluble CR1 was added instead of erythrocytes. This would enable the 7G9 component of the HP to bind to its antigen thereby activating the Fc of the 7G9. If physical clearance alone was the reason for the observed protection with HP and erythrocytes, then addition of soluble CR1 to HPs should not yield any protection. However, this is not the case since significant protection of macrophages incubated with HP 3F3 and soluble CR1 in the presence of Lethal toxin was observed, supporting hypothesis 2.

Materials and Reagents:

Monkey Erythrocytes: cynoblood in alsevers, Lot # 081537770, Cat # B1-160N-03 (#3) from Lampine Bio Labs. Macrophage cells: J774A1, passage #7, viability was 68.4%, passed at 2×10⁶ cells/ml. rPA (1.18 mg/ml), Lot # 149-21 (aliquoted by CF) diluted 1:100 (2 μl aliquot+198 μl DMEM). Lethal factor (LF) (2.92 mg/ml), diluted 1:100 (2 μl aliquot+198 μl DMEM). HP sample: 3F3/7G9, Lot # 159-45, concentration was 970.9 μg/ml. CR1 (soluble): Lot # 013-03 (thawed on Aug. 27, 2002). CR1 stock concentration was 5 mg/ml. Diluted 1:1 0 and then to 5 μg/ml. Shaking speed was 2.1.

Procedure:

1. Diluted HP as in Table 5 (based on molar ratio of PA): 3F3 HP (Lot # 159-45), 970.9 μg/ml.

TABLE 5

| | Final | Working stock | | |
|---|---|---|---|---|
| | Concentration | concentration | | |
| HP 3F3 | (ng/ml) | (μg/ml) | μl of HP | cDMEM |
| 0.5x | 126.3 | 1.01 | 1.04 | 998.96 |
| 0.25x | 63.1 | 0.5048 | 400 of 0.5x | 400 |
| 0.125x | 31.6 | 0.2528 | 400 of 0.25x | 400 |
| 0.0625 | 15.8 | 0.1264 | 400 of 0.125x | 400 |

2. diluted lethal toxin and HP in tubes with Es or with medium;

3. PA working stock concentration: the final concentration of rPA (1.2 mg/ml) in cells is 43.0 ng/ml, stock of PA was 0.012 mg/ml (1:100 dilution). The working stock concentration was 8×43 ng/ml=344 ng/ml, add 86 μl of PA stock (12 μg/ml) to 3 ml of cDMEM;

LF working stock concentration: the final concentration of LF (2.92 mg/ml) in cells was 43.0 ng/ml, the stock of LF was 28.2 μg/ml, the working stock concentration was 8×43 ng/ml=344 ng/ml, added 35.3 μl of LF stock (29.2 μg/ml) to 3 ml cDMEM;

5. Add LeTx/HP or LeTx/Mab mixture to 4×10⁵ J774A.1 cells

6. after a 3 hr incubation, took cells out from the shaker. Washed one time with cold PBS/0.5% BSA buffer;

7. added 200 μl of the staining solution (containing 15 μl of PI stock, 0.5 μl of anti-CD45-FITC and 184.5 μl of buffer).

8. incubated at 4° C. for 20 min. and washed 2 times;

9. added 2 ml of BD FACS lysing solution to all the tubes and incubate at room temperature for 10 min.;

10. washed 2 times with cold buffer and incubated the final pellet in 400 μl of buffer;

11. analyzed on the FACS calibur within 1 hour.

Results:

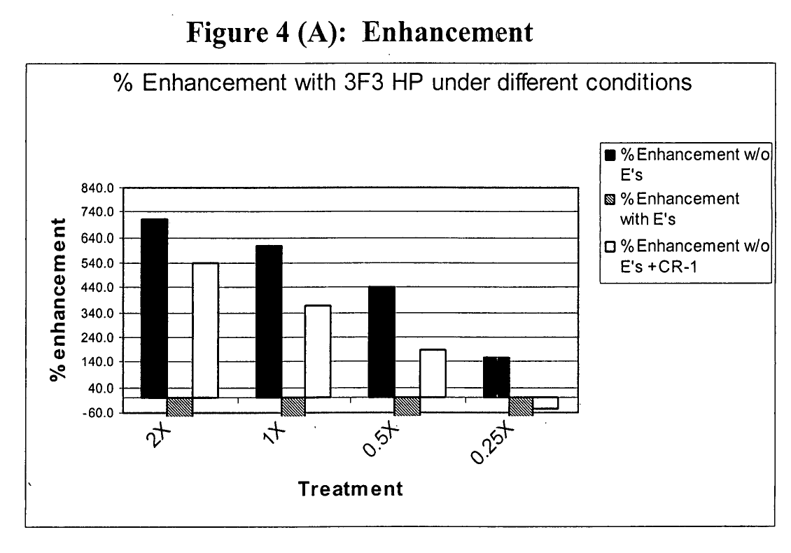

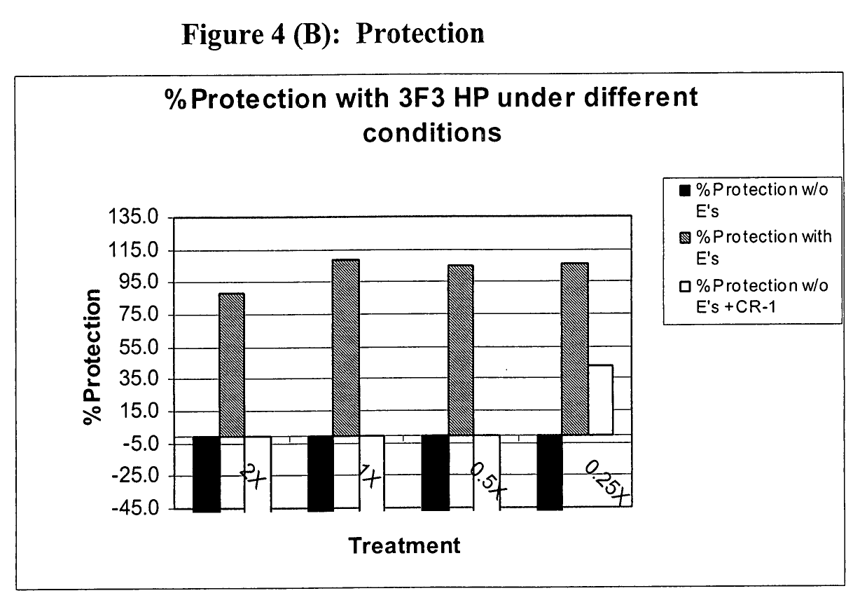

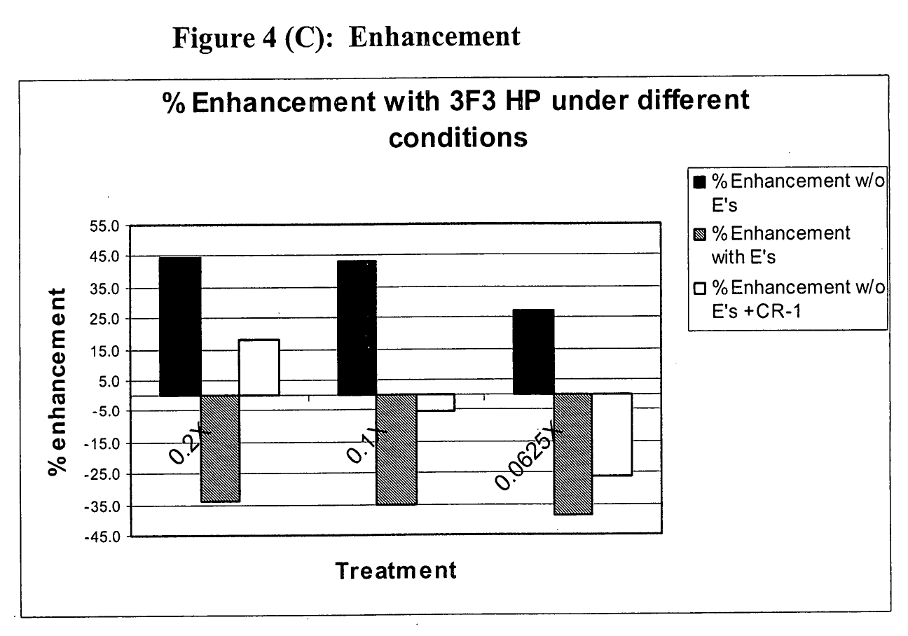

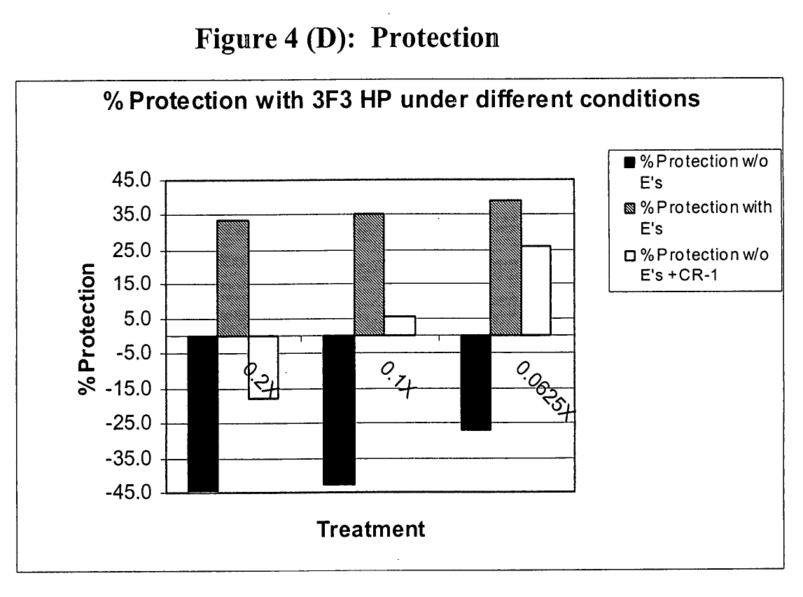

The percentage of enhancement and the percentage of protection of bispecific molecule 7G9 cross-linked to 3F3 in the presence of erythrocytes, or in the presence of soluble CR1, are shown in Table 6, Table7 and FIG. 4.

As can be seen from these data, significant protection of macrophages incubated with bispecific molecule (HP 3F3) and soluble CR1 in the presence of Lethal toxin was observed.

TABLE 6

| | | | | | | | | | | Mean w/ | | | | | | | | |
|---|---|---|---|---|---|---|---|---|---|---|---|---|---|---|---|---|---|---|
| | Set 1 | | | Set 2 | | | Mean | | | background subt. | | | % Enhancement | | | % Protection | | |
| | | | w/o | | | w/o | | | w/o | | | w/o | | | w/o | | | w/o |
| | w/o | with | E's + | w/o | with | E's + | w/o | with | E's + | w/o | with | E's + | w/o | with | E's + | w/o | with | E's + |
| | E's | E's | CR-1 | E's | E's | CR-1 | E's | E's | CR-1 | E's | E's | CR-1 | E's | E's | CR-1 | E's | E's | CR-1 |
| Cells only | 4.10 | 3.04 | 3.51 | 4.76 | 3.93 | 3.69 | 4.43 | 3.49 | 3.60 | 0.0 | 0.0 | 0.0 | | | | | | |
| LeTx | 12.60 | 9.65 | 11.10 | 13.00 | 10.90 | 10.80 | 12.80 | 10.28 | 10.95 | 8.4 | 6.8 | 7.4 | −0.4 | −0.2 | −0.7 | −104.3 | −103.2 | −109.1 |
| 2X | 68.30 | 3.71 | 50.10 | 77.00 | 4.91 | 51.60 | 72.65 | 4.31 | 50.85 | 68.2 | 0.8 | 47.3 | 712.1 | −87.9 | 538.5 | −712.1 | 87.9 | −538.5 |
| 1X | 63.30 | 2.54 | 39.30 | 63.80 | 3.22 | 37.40 | 63.55 | 2.88 | 38.35 | 59.1 | −0.6 | 34.8 | 603.8 | −109.0 | 369.6 | −603.8 | 109.0 | −369.6 |
| 0.5X | 50.70 | 2.83 | 26.80 | 49.00 | 3.49 | 23.40 | 49.85 | 3.16 | 25.10 | 45.4 | −0.3 | 21.5 | 440.7 | −104.9 | 190.5 | −440.7 | 104.9 | −190.5 |
| 0.25X | 27.2 | 3.76 | 11.2 | 25 | 2.44 | 4.4 | 26.1 | 3.1 | 7.8 | 21.7 | −0.4 | 4.2 | 158.0 | −105.7 | −43.2 | −158.0 | 105.7 | 43.2 |

TABLE 7

| | | | | | | | | | | % Mean aft. BG | | | | | | | | |
|---|---|---|---|---|---|---|---|---|---|---|---|---|---|---|---|---|---|---|
| | Set 1 | | | Set 2 | | | Mean Dead | | | subt. | | | % Enhancement | | | % Protection | | |
| | | | w/o | | | w/o | | | w/o | | | w/o | | | w/o | | | w/o |
| | w/o | with | E's + | w/o | with | E's + | w/o | with | E's + | w/o | with | E's + | w/o | with | E's + | w/o | with | E's + |
| | E's | E's | CR-1 | E's | E's | CR-1 | E's | E's | CR-1 | E's | E's | CR-1 | E's | E's | CR-1 | E's | E's | CR-1 |
| Cells | 23.20 | 21.80 | 19.20 | 22.10 | 23.40 | 16.40 | 22.65 | 22.60 | 17.80 | −0.2 | −0.5 | 0.9 | | | | | | |
| only | | | | | | | | | | | | | | | | | | |
| LeTx | 73.90 | 69.70 | 72.60 | 71.00 | 65.80 | 73.70 | 72.45 | 67.75 | 73.15 | 49.6 | 44.6 | 56.3 | 0.0 | 0.0 | −0.1 | −100.0 | −100.0 | −100.2 |
| 0.2X | 96.20 | 54.00 | 85.80 | 92.60 | 51.30 | 81.00 | 94.40 | 52.65 | 83.40 | 71.6 | 29.5 | 66.5 | 44.3 | −33.9 | 18.1 | −44.3 | 33.9 | −18.1 |
| 0.1X | 94.20 | 52.60 | 71.80 | 93.30 | 51.40 | 68.20 | 93.75 | 52.00 | 70.00 | 70.9 | 28.9 | 53.1 | 42.9 | −35.3 | −5.7 | −42.9 | 35.3 | 5.7 |
| 0.0625X | 86.20 | 53.30 | 61.20 | 85.80 | 47.60 | 55.90 | 86.00 | 50.45 | 58.55 | 63.2 | 27.3 | 41.7 | 27.3 | −38.8 | −26.0 | −27.3 | 38.8 | 26.0 |

### Example 4

### Use of Heteropolymers Made Using a Non-Neutralizing Monoclonal Antibody Against Anthrax Protective Antigen in Inactivation of Mutant Forms of Anthrax Toxin

Mutant forms of the Anthrax Protective Antigen (PA) were obtained. Substitution mutations were made in the amino acid sequence of PA. The two mutants that were most potent in cell killing were L685A and K684A (Rosovitz M. J., P. Schuck, M. Varughese, A. P. Chopra, V. Mehra, Y. Singh, L. M. McGinnis, S. H. Leppla. 2003. J Biol Chem. 278:30936). These mutants retain binding to PA receptors on cells but are unable to be neutralized by Monoclonal antibody (Mab) 14B7 or H25 (an affinity enhanced anti-PA Mab derived from 14B7). The mutant toxin is a mixture of mutant PA and LF.

Methods:

Lethal Toxin Cytotoxicity Assay:

Cytotoxicity of anthrax Lethal toxin (LeTx) and the mutant toxins was measured as previously described (Little S. F., S. H. Leppla, A. M Friedlander. 1990. Infect Immun. 58:1606), with some modifications. Wells of 96-well tissue culture microtiter plates were seeded with 10⁵ J774A.1 cells. Toxin components were incubated for 1 hour at 37° C. in a dilution plate prior to addition to macrophages. For neutralization experiments Mab was added to the toxin components for 1 hour at 37° C. The LeTx reaction mixture was added to the macrophages, and after a 4-hour incubation with cells at 37° C., MTT [3-(4,5-dimethylthiazole-2-yl)-2,5-diphenyl tetrazolium bromide] was added and cells incubated for 1 hour at 37° C. Cells were lysed and solubilized by addition of lysing/solubilization buffer (Hansen M. B., S. E. Nielsen, K. Berg. 1989. J Immunol Meth. 119:203). After an overnight incubation at 37° C., the plates were read at 570 nm on a plate reader (SpectraMax 340 pc, Molecular Devices, Sunnyvale Calif.) and the data were analyzed using SoftMaxPro® software (Molecular Devices, Sunnyvale Calif.).

Macrophage Viability Assay:

Cynomologous monkey erythrocytes (Es) were washed and resuspended in Dulbeco's Modified Eagle medium with 5% Fetal Bovine Serum. PA or L685A or K684A at a concentration of 50 ng/ml each were mixed with LF at the same concentration. Various amounts of HP or Mab were added to Es or to medium, and incubated for 1 hour at 37° C. The reaction mixture was then added to J774A.1 macrophages in polystyrene tubes. The tubes were incubated in a CO₂ incubator at 37° C. for 4 hours with constant shaking followed by 2 washes with PBS/BSA buffer. BD FACS Lysing Solution was then added to all the tubes and incubated for 10 minutes at room temperature to lyse Es. The cells were washed twice, and stained with a cocktail of CD45-FITC and propidium iodide (PI) for 20 minutes at room temperature. Cells were then washed twice followed immediately by acquisition of data on the flow cytometer. The CD45 positive population (macrophages) was selectively gated. The proportion of dead cells was determined for each sample by gating on the population positive for PI staining.

Results:

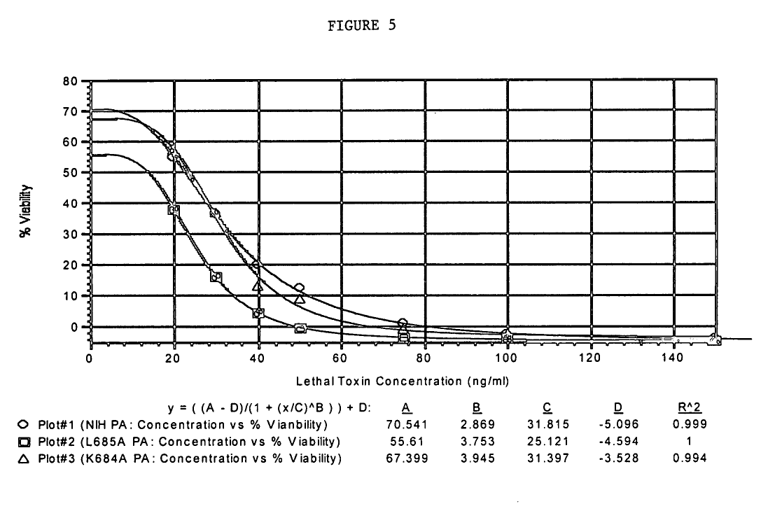

FIG. 4 shows cytotoxicity of RAW 264.7 macrophages using PA, K684A, and L685A in the presence of Lethal Factor. As shown in the figure, the wild-type PA and the K684A and L685A mutant forms were toxic to macrophages. FIG. 5 shows neutralization of anthrax Lethal toxin (PA+LF) with Mab H25 alone. As shown in the figure, the mutant toxins (L685A+LF, K684A+LF) were not neutralized with anti-PA Mab H25.

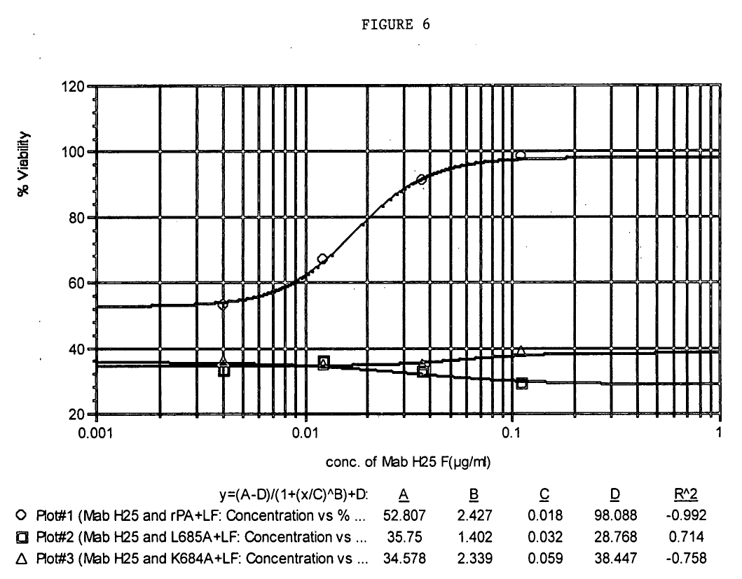

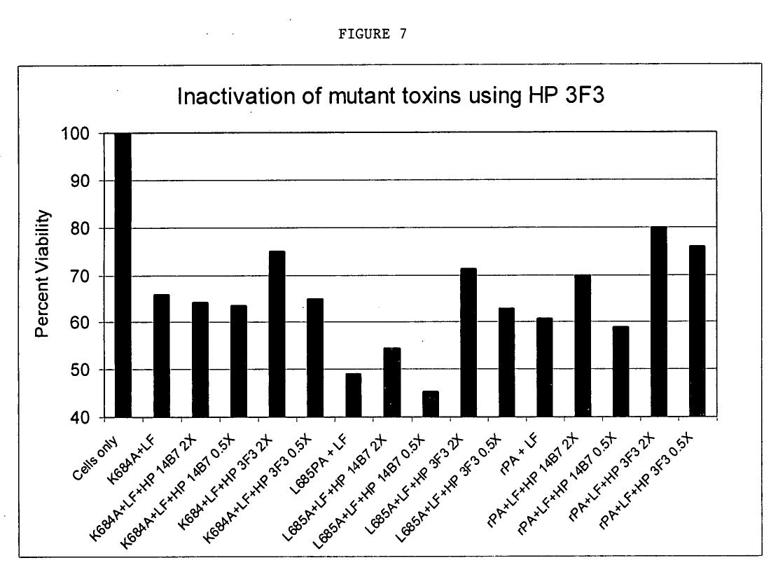

HP was then made using non-neutralizng antibody Mab3F3. FIG. 6 shows inactivation of mutant anthrax toxin by HP made using this non-neutralizing antibody. HP was also made using Mab 14B7 (which is a neutralizing Mab) and is ineffective in inactivating the mutant toxins

### Example 5

### Use of Heteropolymers Made Using a Non-Neutralizing Monoclonal Antibody Against Staphylococcus Aureus for Inactivation of the Pathogen

An animal model for lethal challenge with S. aureus will be developed. This model will be used to test our hypothesis that an HP made using a non-neutralizing Mab will be able to inactivate its target pathogen, S. aureus. The anti-S. aureus Mab to be used is an anti-Protein A Mab (Catalog # P 2921, Sigma Aldrich, St, Louis Mo.). This Mab is likely to be non-neutralizing since Protein A is not known to be involved with binding to any surface proteins in animals or humans. A Heteropolymer (HP) made by cross-linking the anti-protein A Mab to the anti-complement receptor type 1 (CR1) Mab 7G9 will clear the S. aureus to the erythrocyte (E) surface. Based on previous models of HP action, the E:HP:S. aureus complexes will be cleared to the fixed tissue macrophages (Kuppfer cells) in the liver where the immune complex (CR1:HP:S. aureus) will be destroyed. On the other hand, Mab alone will not be as effective in protecting the mice from a lethal S. aureus challenge since (i) Protein A is not involved in tissue invasion and (ii) density of protein A on the surface of the organism is relatively high and all the protein A on the surface may not blocked by the Mab. In contrast to Mab alone, in order for the HP to be effective, there is no need for all the protein A to be bound since a few HPs can tether the microorganism to the E and inactivate the pathogen.

Methods:

The aim of this experiment is to determine the efficacy of HP versus Mab at preventing death in CR1 transgenic mice injected with S. aureus. CR1 mice will be injected with either PBS, Mab or HP IV followed by S. aureus IV. The groups sizes will be 10 mice/group.

Stock cultures of S. aureus will be prepared, aliquoted and frozen at −80 degrees C. Defrosted bacteria will be titered in advance. On the day of injection, bacteria will be diluted for injection and re-titered. Animals (e.g., mice) will be injected with saline, HPs or Mabs in a total volume of 100 μl IV. One hour later inject S. aureus in a total volume of 100 μl IV. Animals will be monitored for 21 days post-injection or until death. Animals will be monitored twice daily for time to death (TTD) for 21 days.

Animals that are moribund will be euthanized. A summary of the experimental design is shown in Table 8.

TABLE 8
Study in an animal model to determine the efficacy of HP versus
Mab in protection against a lethal S. aureus challenge.

| Group ID | # animals | Treatment | Manipulation |
|---|---|---|---|
| 1 | 10 | Saline | S. aureus (˜3xLD100) injected IV |
| | | | after saline |
| 2 | 10 | 20 μg anti-S. aureus HP | HP injected IV 1 hour prior to S. aureus |
| | | (7G9 X anti-protein A Mab) | (˜3xLD100) injected IV |
| 3 | 10 | 10 μg anti-S. aureus Mab | Mab injected IV 1 hour prior to S. aureus |
| | | (Anti-protein A Mab) | (˜3xLD100) injected IV |
| 4 | 10 | 5 μg anti-S. aureus HP | HP injected IV 1 hour prior to S. aureus |
| | | (7G9 X anti-protein A Mab) | (˜3xLD100) injected IV |
| 5 | 10 | 2.5 μg anti-S. aureus Mab | Mab injected IV 1 hour prior to S. aureus |
| | | (Anti-protein A Mab) | (˜3xLD100) injected IV |
| 6 | 10 | 20 μg control HP | HP injected IV 1 hour prior to S. aureus |
| | | (7G9 X anti-PA Mab14B7) | (˜3xLD100) injected IV |
| 7 | 10 | 10 μg anti-CR1 Mab 7G9 | Mab injected IV 1 hour prior to S. aureus |
| | | | (˜3xLD100) injected IV |

### References Cited and Equivalents

All references cited herein (including, e.g., books, journal articles, issued patents, and patent applications) are incorporated herein by reference in their entirety and for all purposes to the same extent as if each individual publication or patent or patent application was specifically and individually indicated to be incorporated by reference in its entirety for all purposes.

Many modifications and variations of this invention can be made without departing from its spirit and scope, as will be apparent to those skilled in the art. The specific embodiments described herein are offered by way of example only, and the invention is to be limited only by the terms of the appended claims along with the full scope of equivalents to which such claims are entitled.

1. A bispecific molecule comprising an anti-CR1 antibody linked to a non-neutralizing antibody that binds a pathogenic agent of an animal.

2. The bispecific molecule of claim 1, wherein the non-neutralizing antibody is an enhancing antibody.

3. The bispecific molecule of claim 1, wherein the anti-CR1 antibody is cross-linked to the non-neutralizing antibody that binds the pathogenic agent.

4. The bispecific molecule of claim 1, wherein the pathogenic agent is a bacterium.

5. The bispecific molecule of claim 1, wherein the pathogenic agent is a virus.

6. The bispecific molecule of claim 1, wherein the pathogenic agent is a microbial toxin.

7. The bispecific molecule of claim 1, wherein at least one of the anti-CR1 antibody and the non-neutralizing antibody are monoclonal antibodies.

8. The bispecific molecule of claim 1, wherein one or more of the antibodies is modified to reduce its immunogenicity.

9. The bispecific molecule of claim 8, wherein one or more of the antibodies is deimmunized.

10. The bispecific molecule of claim 1, wherein the first and second antibody are crosslinked using a crosslinking agent.

11. The bispecific molecule of claim 10, wherein the crosslinking agent is polyethylene glycol (PEG).

12. The bispecific molecule of claim 1, wherein the anti-CR1 antibody is 7G9.

13. The bispecific molecule of claim 1, wherein the anti-CR1 antibody is 19E9.

14. The bispecific molecule of claim 1, wherein the non-neutralizing antibody binds a protective antigen (PA) of a Bacillus anthracis toxin.

15. The bispecific molecule of claim 14, wherein the non-neutralizing antibody is 3F3.

16. The bispecific molecule of claim 15, wherein the anti-CR1 antibody is selected from the group consisting of: 7G9 and 19E9.

17. The bispecific molecule of claim 1, wherein the non-neutralizing antibody binds S. aureus.

18. The bispecific molecule of claim 17, wherein the non-neutralizing antibody binds protein A.

19. A bispecific molecule comprising an anti-CR1 antibody linked to an antibody that is selected from the group consisting of: 3F3, 2F9, 3F10, 3D2, 16E11, 2C11, 6C3, and an antibody that recognizes protein A.

20. A bispecific molecule comprising a first antibody that binds a CR1 receptor coupled to a second antibody that binds to a protective antigen component of anthrax toxin but does not inhibit the binding of the protective antigen component of the anthrax toxin to cells.

21. A method of treating or preventing a disease associated with presence of a pathogenic agent of an animal in the circulation of a subject, comprising administering to the subject a therapeutically or prophylactically effective amount of a bispecific molecule comprising an anti-CR1 antibody linked to a non-neutralizing antibody that binds to the pathogenic agent.

22. The method of claim 21, wherein the non-neutralizing antibody is an enhancing antibody.

23. The bispecific molecule of claim 21, wherein the first and second antibody are crosslinked using a crosslinking agent.

24. The bispecific molecule of claim 23, wherein the crosslinking agent is polyethylene glycol (PEG).

25. The method of claim 21, wherein one or more of the antibodies is a monoclonal antibody.

26. The method of claim 21, wherein one or more of the antibodies is modified to reduce its immunogenicity.

27. The method of claim 21, wherein the subject is a human.

28. The method of claim 21, wherein the anti-CR1 antibody is selected from the group consisting of: 7G9 and 19E9.

29. A method of treating or preventing bacterial infection in a subject, comprising administering to the subject a therapeutically or prophylactically effective amount of a bispecific molecule comprising an anti-CR1 antibody linked to a non-neutralizing antibody that binds to a bacterium.

30. The method of claim 29, wherein the bacterium is a gram negative bacterium.

31. The method of claim 29, wherein the bacterium is a gram positive bacterium.

32. The method of claim 31, wherein the bacterium is S. aureus.

33. The method of claim 29, wherein the non-neutralizing antibody is an enhancing antibody.

34. The method of claim 29, wherein the anti-CR1 antibody is cross-linked to the non-neutralizing antibody that binds the bacterium.

35. The method of claim 29, wherein the anti-CR1 antibody and the non-neutralizing antibody are monoclonal antibodies.

36. The method of claim 29, wherein the subject is a human.

37. The method of claim 29, wherein the anti-CR1 antibody is selected from the group consisting of: 7G9 and 19E9.

38. The method of claim 32, wherein the non-neutralizing antibody is an antibody that recognizes protein A.

39. The method of claim 38, wherein the anti-CR1 antibody is selected from the group consisting of: 7G9 and 19E9.

40. A method of treating or preventing a viral infection in an animal subject, comprising administering to the subject a therapeutically or prophylactically effective amount of a bispecific molecule comprising an anti-CR1 antibody linked to a non-neutralizing antibody that binds an epitope of the virus.

41. The method of claim 40, wherein the antibody binds to an envelope (E) protein of the virus.

42. The method of claim 40, wherein the non-neutralizing antibody is an enhancing antibody.

43. The method of claim 40, wherein one or more of the antibodies is a monoclonal antibody.

44. The method of claim 40, wherein the subject is a human.

45. The method of claim 21, wherein the anti-CR1 antibody is selected from the group consisting of: 7G9 and 19E9.

46. A method of prophylactically preventing or reducing the symptoms of exposure to anthrax spores comprising, administering a bispecific molecule comprising a first antibody that recognizes a C3b receptor coupled to a second antibody that binds to a protective antigen component of anthrax toxin but does not inhibit the binding of the protective antigen component of the anthrax toxin to cells, to a subject at risk of exposure to anthrax spores to thereby prevent or reduce the symptoms of exposure to anthrax spores.

47. The method of claim 46, wherein the C3b receptor is CR1.

48. The method of claim 46, wherein one or more of the antibodies is modified to reduce its immunogenicity.

49. The method of claim 46, wherein one or more of the antibodies is a monoclonal antibody.

50. The bispecific molecule of claim 46, wherein the first and second antibody are crosslinked using a crosslinking agent.

51. The bispecific molecule of claim 46, wherein the crosslinking agent is polyethylene glycol (PEG).

52. The method of claim 46, wherein the anthrax toxin is a mutant form that does not bind to antibodies that inhibit the binding of the protective antigen component of the toxin to cells.

53. The method of claim 46, wherein the antibody that binds to a protective antigen component of anthrax toxin is selected from the group consisting of: 3F3, 2F9, 3F10, 3D2, 16E11, 2C11 and 6C3.

54. A method of reducing the symptoms of exposure to anthrax spores in a population, comprising, administering a bispecific molecule comprising a first antibody that recognizes a C3b receptor coupled to a second antibody that binds to a protective antigen component of anthrax toxin but does not inhibit the binding of the protective antigen component of the anthrax toxin to cells, to multiple subjects at risk of exposure to anthrax spores to thereby prevent or reduce the symptoms of exposure to anthrax spores.

55. A method of therapeutically treating the symptoms of exposure to anthrax spores comprising, administering a bispecific molecule comprising a first antibody that recognizes a C3b receptor coupled to a second antibody that binds to a protective antigen component of anthrax toxin but does not inhibit the binding of the protective antigen component of the anthrax toxin to cells, to a subject exposed to anthrax spores to thereby prevent or reduce the symptoms of exposure to anthrax spores.

56. The method of claim 54 or 55, wherein the C3b receptor is CR1.

57. The method of claim 54 or 55, wherein one or more of the antibodies is modified to reduce its immunogenicity.

58. The bispecific molecule of claim 54 or 55, wherein the first and second antibody are crosslinked using a crosslinking agent.

59. The bispecific molecule of claim 58, wherein the crosslinking agent is polyethylene glycol (PEG).

60. The method of claim 54 or 55, wherein the anthrax toxin is a mutant form that does not bind to antibodies that inhibit the binding of the protective antigen component of the toxin to cells.

61. The method of claim 54 or 55, wherein the antibody that binds to a protective antigen component of anthrax toxin is selected from the group consisting of: 3F3, 2F9, 3F10, 3D2, 16E11, 2C11 and 6C3.

62. A method of enhancing the protective effect of a non-neutralizing antibody that binds a pathogenic agent of an animal, comprising linking the antibody to a second antibody that binds to CR1.
